(19) United States
(12) Patent Application Publication
(10) Pub. No.: US 2025/0197514 A1
(43) Pub. Date: Jun. 19, 2025

(54) ANTIBODIES AND IgG FUSION PROTEINS WITH AN EXTENDED HALF-LIFE

(71) Applicant: Intervet Inc., Rahway, NJ (US)

(72) Inventors: Mohamad Morsey, Omaha, NE (US); Seth D. Stauffer, Elkhorn, NE (US)

(73) Assignee: Intervet Inc., Rahway, NJ (US)

(21) Appl. No.: 18/684,156

(22) PCT Filed: Aug. 19, 2022
(86) PCT No.: PCT/EP2022/073145

§ 371 Date: Feb. 15, 2024

Related U.S. Application Data
(60) Provisional application No. 63/341,443, filed on May 13, 2022.
(60) Provisional application No. 63/235,259, filed on Aug. 20, 2021.
(60) Provisional application No. 63/235,261, filed on Aug. 20, 2021.

(51) Int. Cl. C07K 16/28; C07K 14/715

(52) U.S. Cl. CPC C07K 16/2866; C07K 14/7155; C07K 2317/20; C07K 2317/24; C07K 2317/53; C07K 2317/92; C07K 2317/94; C07K 2319/30

(57) ABSTRACT

The present invention provides antibodies or IgG Fc fusion proteins comprising a canine or feline fragment crystallizable region (Fc region) that has an enhanced half-life due to one or more specific amino acid substitutions in the Fc region. The increased half-life can be a function of an increased binding affinity for a neonatal Fc receptor (FcRn) at moderately acidic pH relative to that of corresponding antibody or IgG Fc fusion protein that comprises an unsubstituted canine or feline Fc region. Pharmaceutical compositions that comprise these antibodies and/or IgG fusion proteins are also provided.

### SEQUENCE LISTING

This application contains an electronic Sequence Listing which has been submitted in XML file format via Patent Center, the entire content of which is incorporated by reference herein in its entirety. The Sequence Listing XML file submitted via Patent Center is entitled “14463-300-999_SUB_SEQ_LISTING.xml”, was created on Sep. 16, 2024, and is 86,175 bytes in size.

### CROSS-REFERENCE TO RELATED APPLICATIONS

This application is a national stage application of International Patent Application No. PCT/EP2022/073145 filed Aug. 19, 2022, which claims priority under 35 U.S.C. § 119(e) of U.S. Provisional Patent Application No. 63/341,443, filed on May 13, 2022, U.S. Provisional Patent Application No. 63/235,259, filed Aug. 20, 2021 and U.S. Provisional Patent Application No. 63/235,261, filed Aug. 20, 2021, each of which is incorporated by reference in its entirety herein.

### FIELD OF THE INVENTION

The present invention relates to antibodies or IgG Fc fusion proteins comprising a canine or feline fragment crystallizable region (Fc region) that has an enhanced half-life due to one or more specific amino acid substitutions in the Fc region. The increased half-life can be a function of an increased binding affinity for a neonatal Fc receptor (FcRn) at moderately acidic pH relative to that of corresponding antibody or IgG Fc fusion protein that comprises an unsubstituted canine or feline Fc region. Pharmaceutical compositions that comprise these antibodies and/or IgG fusion proteins are also provided.

### BACKGROUND OF THE INVENTION

One important goal of pharmaceutical antibody research is to develop antibodies that are effective at relatively lower doses and reduced dosing frequency. These attributes serve to lower the cost of treatment, which leads to greater patient access while at the same time increasing patient convenience and boosting patient compliance, thereby resulting in a better therapeutic outcome. In order to achieve this goal many studies focused on modulating the binding between therapeutic antibodies of the IgG isotype and the so-called neonatal Fc receptor (FcRn). This is because FcRn binding to IgG is considered a key factor in maintaining and extending antibody plasma half-life.

The FcRn is a heterodimer composed of the MHC class I-like alpha domain and the B2-microglobulin (B2-m) subunits. FcRn is expressed in several tissues: most notably in the vascular endothelium, kidneys, bone-marrow derived cells, and the blood-brain barrier. FcRn binds to IgG at a site in the IgG Fc that is distinct from the sites for the other IgG Fc receptors. Binding of IgG to FcRn is highly dependent on pH and this binding occurs with high affinity at low pH (e.g., below pH 6.5) in endosomal compartments, but with a significant lower binding affinity at physiological pH (e.g., pH 7.4) at the cell surface [see, e.g., Borok et al., J. Biol. Chem. 290 (7): 4282-4290, (2015)]. The strong binding of IgG to FcRn in endosomal compartment protects the antibody from degradation by proteolytic enzymes in endosomes and allows recycling of the receptor-bound antibody to the cell surface where the increased pH weakens the interaction and allow release of the antibody into circulation at physiological pH.

Previous studies showed certain mutations at certain positions alter the binding of human IgG to human FcRn [see e.g., U.S. Pat. Nos. 7,083,784, 7,658,921, 7,217,797, 7,217,798, 8,088,376, 10,336,818 and WO2019/147973]. However, the human and dog or cat IgG proteins have many differences in their amino acids sequences which give the residues in the human sequence different environments and/or different identities than in the dog or cat sequence. This variability makes it difficult to directly transfer characteristics of one IgG from one species to an IgG from another species. Consistently, a number of corresponding studies have more recently been conducted on companion animals [see, e.g., WO2018073185, WO2020082048, WO20210116560, US20200362035, US20200216536, WO2020191289, WO2021231464, and US20210347854]. However, any particular mutation or combination of mutations may have drastically different effects on prolongation of half-life or other attributes of antibodies and this makes it also difficult to predict the effect of such mutations on key antibody properties including the potential to induce anti-drug (antibody) antibodies. Therefore, there still remains a need to identify new specific substitutions/mutations in the dog and/or cat IgG proteins that can extend the half-life of therapeutic antibodies and/or corresponding therapeutic Fc fusion proteins.

The citation of any reference herein should not be construed as an admission that such reference is available as “prior art” to the instant application.

### SUMMARY OF THE INVENTION

The present invention provides antibodies and IgG Fe fusion proteins with enhanced half-lives. Accordingly, in one aspect of the present invention an antibody that comprises a light chain and a heavy chain is provided, in which the heavy chain comprises a fragment crystallizable region (Fc region) comprising an amino acid substitution at an amino acid residue position numbered according to the EU index as in Kabat at amino acid residue position 252, at amino acid residue position 254, at amino acid residue position 256, at amino acid residue position 308, at amino acid residue position 433, at amino acid residue position 434, at amino acid residue position 436, or at any combination of these amino acid positions. In certain embodiments of this type the Fc region is a canine Fc region (cFc). In other embodiments of this type the Fc region is a feline Fc region (fFc).

The present invention also provides IgG Fc fusion protein that comprises a fragment crystallizable region (Fc region) comprising an amino acid substitution at an amino acid residue position numbered according to the EU index as in Kabat at amino acid residue position 252, at amino acid residue position 254, at amino acid residue position 256, at amino acid residue position 308, at amino acid residue position 433, at amino acid residue position 434, at amino acid residue position 436, or at any combination of these amino acid positions. In certain embodiments of this type the Fc region is a canine Fc region (cFc). In other embodiments of this type the Fc region is a feline Fc region (fFc).

In particular embodiments, the antibodies or IgG Fc fusion proteins of the invention comprise one or more such amino acid substitutions. In certain embodiments, a substitution is with a tyrosine residue at amino acid residue position 252. In other embodiments, a substitution is with a threonine residue at amino acid residue position 254. In yet other embodiments, a substitution is with an aspartic acid residue at amino acid residue position 256. In still other embodiments, a substitution is with a glutamic acid residue at amino acid residue position 256. In yet other embodiments, a substitution is with a proline residue at amino acid residue position 308. In still other embodiments, a substitution is with a lysine residue at amino acid residue position 433. In yet other embodiments, a substitution is with a leucine residue at amino acid residue position 433. In still other embodiments, a substitution is with a phenylalanine residue at amino acid residue position 434. In yet other embodiments, a substitution is with a histidine residue at amino acid residue position 434. In still other embodiments, a substitution is with a tyrosine residue at amino acid residue position 434. In yet other embodiments, a substitution is with a threonine residue at amino acid residue position 436.

In related embodiments, the antibodies or IgG Fc fusion proteins of the invention comprise two or more of such amino acid substitutions. In particular embodiments, the antibodies or IgG Fc fusion proteins of the invention comprise a substitution with a tyrosine residue at amino acid residue position 252 and a substitution with an aspartic acid residue at amino acid residue position 256. In other embodiments, a substitution is with a proline residue at amino acid residue position 308 and a substitution is with a tyrosine residue at amino acid residue position 434. In yet other embodiments, a substitution is with a lysine residue at amino acid residue position 433 and a substitution is with a phenylalanine residue at amino acid residue position 434. In still other embodiments, a substitution is with a lysine residue at amino acid residue position 433 and a substitution is with a tyrosine residue at amino acid residue position 434. In yet other embodiments, a substitution is with a leucine residue at amino acid residue position 433 and a substitution is with a phenylalanine residue at amino acid residue position 434. In still other embodiments, a substitution is with a leucine residue at amino acid residue position 433 and a substitution is with a tyrosine residue at amino acid residue position 434. In yet other embodiments, a substitution is with an aspartic acid residue at amino acid residue position 256 and a substitution is with a tyrosine residue at amino acid residue position 434. In still other embodiments, a substitution is with a tyrosine residue at amino acid residue position 434 and a substitution is with a threonine residue at amino acid residue position 436. In yet other embodiments, a substitution is with a tyrosine residue at amino acid residue position 252, a substitution is with a threonine residue at amino acid residue position 254, and a substitution is with a glutamic acid residue at amino acid residue position 256. In still other embodiments, a substitution is with an aspartic acid residue at amino acid residue position 256, a substitution is with a proline residue at amino acid residue position 308, and a substitution is with a tyrosine residue at amino acid residue position 434. In yet other embodiments, a substitution is with a lysine residue at amino acid residue position 433, a substitution is with a phenylalanine residue at amino acid residue position 434, and a substitution is with a threonine residue at amino acid residue position 436. In still other embodiments, a substitution is with a lysine residue at amino acid residue position 433, a substitution is with a tyrosine residue at amino acid residue position 434, and a substitution is with a threonine residue at amino acid residue position 436. In yet other embodiments, a substitution is with a leucine residue at amino acid residue position 433, a substitution is with a phenylalanine residue at amino acid residue position 434, and a substitution is with a threonine residue at amino acid residue position 436. In still other embodiments, a substitution is with a leucine residue at amino acid residue position 433, a substitution is with a tyrosine residue at amino acid residue position 434, and a substitution is with a threonine residue at amino acid residue position 436.

The antibodies and the IgG Fc fusion proteins of the invention preferably have an increased half-life compared to the half-life of the corresponding antibody or the corresponding IgG Fc fusion protein that comprises the corresponding wild type canine Fc or feline Fc. In particular embodiments, the antibody or the IgG Fc fusion protein has an enhanced binding affinity for their neonatal Fc receptor (FcRn) at moderately acidic pH than the corresponding antibody or the corresponding IgG Fc fusion protein that comprises the corresponding wild type canine Fc or feline Fc.

In certain embodiments of the antibodies or IgG Fc fusion proteins of the invention, the Fc region is a feline Fc region (fFc). In particular embodiments, the fFc is an IgG-1a Fc. In yet other embodiments, the fFc is an IgG-1am Fc. In still other embodiments, the fFc is an IgG-1b Fc. In yet other embodiments, the fFc is an IgG-1bm Fc. In still other embodiments, the fFc is an IgG-2 Fc. In yet other embodiments, the fFc is an IgG-2m Fc.

In more particular embodiments of the antibodies or IgG Fc fusion proteins of the invention, the fFc comprises an amino acid sequence that has at least 90% identity to SEQ ID NO: 9. In other embodiments, the fFc comprises an amino acid sequence that has at least 95% identity to SEQ ID NO: 9. In yet other embodiments, the fFc comprises an amino acid sequence that has at least 97% identity to SEQ ID NO: 9. In still other embodiments, the fFc comprises an amino acid sequence that has at least 98% identity to SEQ ID NO: 9. In yet other embodiments, the fFc comprises an amino acid sequence that has at least 99% identity to SEQ ID NO: 9. In specific embodiments, the fFc comprises the amino acid sequence of SEQ ID NO: 9.

In other particular embodiments of the antibodies or IgG Fc fusion proteins of the invention the fFc comprises an amino acid sequence that has at least 90% identity to SEQ ID NO: 10. In other embodiments, the fFc comprises an amino acid sequence that has at least 95% identity to SEQ ID NO: 10. In yet other embodiments, the fFc comprises an amino acid sequence that has at least 97% identity to SEQ ID NO: 10. In still other embodiments, the fFc comprises an amino acid sequence that has at least 98% identity to SEQ ID NO: 10. In yet other embodiments, the fFc comprises an amino acid sequence that has at least 99% identity to SEQ ID NO: 10. In specific embodiments, the fFc comprises the amino acid sequence of SEQ ID NO: 10.

In still other particular embodiments of the antibodies or IgG Fc fusion proteins of the invention the fFc comprises an amino acid sequence that has at least 90% identity to SEQ ID NO: 11. In other embodiments, the fFc comprises an amino acid sequence that has at least 95% identity to SEQ ID NO: 11. In yet other embodiments, the fFc comprises an amino acid sequence that has at least 97% identity to SEQ ID NO: 11. In still other embodiments, the fFc comprises an amino acid sequence that has at least 98% identity to SEQ ID NO: 11. In yet other embodiments, the fFc comprises an amino acid sequence that has at least 99% identity to SEQ ID NO: 11. In specific embodiments, the fFc comprises the amino acid sequence of SEQ ID NO: 11.

In yet other particular embodiments of the antibodies or IgG Fc fusion proteins of the invention the fFc comprises an amino acid sequence that has at least 90% identity to SEQ ID NO: 12. In other embodiments, the fFc comprises an amino acid sequence that has at least 95% identity to SEQ ID NO: 12. In yet other embodiments, the fFc comprises an amino acid sequence that has at least 97% identity to SEQ ID NO: 12. In still other embodiments, the fFc comprises an amino acid sequence that has at least 98% identity to SEQ ID NO: 12. In yet other embodiments, the fFc comprises an amino acid sequence that has at least 99% identity to SEQ ID NO: 12. In specific embodiments, the fFc comprises the amino acid sequence of SEQ ID NO: 12.

In still other particular embodiments of the antibodies or IgG Fc fusion proteins of the invention the fFc comprises an amino acid sequence that has at least 90% identity to SEQ ID NO: 50. In other embodiments, the fFc comprises an amino acid sequence that has at least 95% identity to SEQ ID NO: 50. In yet other embodiments, the fFc comprises an amino acid sequence that has at least 97% identity to SEQ ID NO: 50. In still other embodiments, the fFc comprises an amino acid sequence that has at least 98% identity to SEQ ID NO: 50. In yet other embodiments, the fFc comprises an amino acid sequence that has at least 99% identity to SEQ ID NO: 50. In specific embodiments, the fFc comprises the amino acid sequence of SEQ ID NO: 50.

In yet other particular embodiments of the antibodies or IgG Fc fusion proteins of the invention the fFc comprises an amino acid sequence that has at least 90% identity to SEQ ID NO: 51. In other embodiments, the fFc comprises an amino acid sequence that has at least 95% identity to SEQ ID NO: 51. In yet other embodiments, the fFc comprises an amino acid sequence that has at least 97% identity to SEQ ID NO: 51. In still other embodiments, the fFc comprises an amino acid sequence that has at least 98% identity to SEQ ID NO: 51. In yet other embodiments, the fFc comprises an amino acid sequence that has at least 99% identity to SEQ ID NO: 51. In specific embodiments, the fFc comprises the amino acid sequence of SEQ ID NO: 51.

In certain embodiments of the antibodies or IgG Fc fusion proteins of the invention, the Fc region is a canine Fc region (cFc). In particular embodiments, the cFc is an IgG-A Fc. In yet other embodiments, the cFc is an IgG-Am Fc. In still other embodiments, the cFc is an IgG-B Fc. In yet other embodiments, the cFc is an IgG-Bm Fc. In still other embodiments, the cFc is an IgG-C Fc. In yet other embodiments, the cFc is an IgG-Cm Fc. In still other embodiments, the cFc is an IgG-D Fc. In yet other embodiments, the cFc is an IgC-Dm Fc.

In more particular embodiments of the antibodies or IgG Fc fusion proteins of the invention, the cFc comprises an amino acid sequence that has at least 90% identity to SEQ ID NO: 1. In other embodiments, the cFc comprises an amino acid sequence that has at least 95% identity to SEQ ID NO: 1. In yet other embodiments, the cFc comprises an amino acid sequence that has at least 97% identity to SEQ ID NO: 1. In still other embodiments, the cFc comprises an amino acid sequence that has at least 98% identity to SEQ ID NO: 1. In yet other embodiments, the cFc comprises an amino acid sequence that has at least 99% identity to SEQ ID NO: 1. In specific embodiments, the cFc comprises the amino acid sequence of SEQ ID NO: 1.

In other particular embodiments of the antibodies or IgG Fc fusion proteins of the invention, the cFc comprises an amino acid sequence that has at least 90% identity to SEQ ID NO: 2. In other embodiments, the cFc comprises an amino acid sequence that has at least 95% identity to SEQ ID NO: 2. In yet other embodiments, the cFc comprises an amino acid sequence that has at least 97% identity to SEQ ID NO: 2. In still other embodiments, the cFc comprises an amino acid sequence that has at least 98% identity to SEQ ID NO: 2. In yet other embodiments, the cFc comprises an amino acid sequence that has at least 99% identity to SEQ ID NO: 2. In specific embodiments, the cFc comprises the amino acid sequence of SEQ ID NO: 2.

In still other particular embodiments of the antibodies or IgG Fc fusion proteins of the invention, the cFc comprises an amino acid sequence that has at least 90% identity to SEQ ID NO: 3. In other embodiments, the cFc comprises an amino acid sequence that has at least 95% identity to SEQ ID NO: 3. In yet other embodiments, the cFc comprises an amino acid sequence that has at least 97% identity to SEQ ID NO: 3. In still other embodiments, the cFc comprises an amino acid sequence that has at least 98% identity to SEQ ID NO: 3. In yet other embodiments, the cFc comprises an amino acid sequence that has at least 99% identity to SEQ ID NO: 3. In specific embodiments, the cFc comprises the amino acid sequence of SEQ ID NO: 3.

In yet other particular embodiments of the antibodies or IgG Fc fusion proteins of the invention, the cFc comprises an amino acid sequence that has at least 90% identity to SEQ ID NO: 4. In other embodiments, the cFc comprises an amino acid sequence that has at least 95% identity to SEQ ID NO: 4. In yet other embodiments, the cFc comprises an amino acid sequence that has at least 97% identity to SEQ ID NO: 4. In still other embodiments, the cFc comprises an amino acid sequence that has at least 98% identity to SEQ ID NO: 4. In yet other embodiments, the cFc comprises an amino acid sequence that has at least 99% identity to SEQ ID NO: 4. In specific embodiments, the cFc comprises the amino acid sequence of SEQ ID NO: 4.

In still other particular embodiments of the antibodies or IgG Fc fusion proteins of the invention, the cFc comprises an amino acid sequence that has at least 90% identity to SEQ ID NO: 5. In other embodiments, the cFc comprises an amino acid sequence that has at least 95% identity to SEQ ID NO: 5. In yet other embodiments, the cFc comprises an amino acid sequence that has at least 97% identity to SEQ ID NO: 5. In still other embodiments, the cFc comprises an amino acid sequence that has at least 98% identity to SEQ ID NO: 5. In yet other embodiments, the cFc comprises an amino acid sequence that has at least 99% identity to SEQ ID NO: 5. In specific embodiments, the cFc comprises the amino acid sequence of SEQ ID NO: 5.

In yet other particular embodiments of the antibodies or IgG Fc fusion proteins of the invention, the cFc comprises an amino acid sequence that has at least 90% identity to SEQ ID NO: 6. In other embodiments, the cFc comprises an amino acid sequence that has at least 95% identity to SEQ ID NO: 6. In yet other embodiments, the cFc comprises an amino acid sequence that has at least 97% identity to SEQ ID NO: 6. In still other embodiments, the cFc comprises an amino acid sequence that has at least 98% identity to SEQ ID NO: 6. In yet other embodiments, the cFc comprises an amino acid sequence that has at least 99% identity to SEQ ID NO: 6. In specific embodiments, the cFc comprises the amino acid sequence of SEQ ID NO: 6.

In still other particular embodiments of the antibodies or IgG Fc fusion proteins of the invention, the cFc comprises an amino acid sequence that has at least 90% identity to SEQ ID NO: 7. In other embodiments, the cFc comprises an amino acid sequence that has at least 95% identity to SEQ ID NO: 7. In yet other embodiments, the cFc comprises an amino acid sequence that has at least 97% identity to SEQ ID NO: 7. In still other embodiments, the cFc comprises an amino acid sequence that has at least 98% identity to SEQ ID NO: 7. In yet other embodiments, the cFc comprises an amino acid sequence that has at least 99% identity to SEQ ID NO: 7. In specific embodiments, the cFc comprises the amino acid sequence of SEQ ID NO: 7.

In yet other particular embodiments of the antibodies or IgG Fc fusion proteins of the invention, the cFc comprises an amino acid sequence that has at least 90% identity to SEQ ID NO: 8. In other embodiments, the cFc comprises an amino acid sequence that has at least 95% identity to SEQ ID NO: 8. In yet other embodiments, the cFc comprises an amino acid sequence that has at least 97% identity to SEQ ID NO: 8. In still other embodiments, the cFc comprises an amino acid sequence that has at least 98% identity to SEQ ID NO: 8. In yet other embodiments, the cFc comprises an amino acid sequence that has at least 99% identity to SEQ ID NO: 8. In specific embodiments, the cFc comprises the amino acid sequence of SEQ ID NO: 8.

In related embodiments of the antibodies or IgG Fc fusion proteins of the invention, the cFc further comprises a canine hinge region. In certain embodiments the canine hinge region comprises an amino acid sequence that has at least 80% identity to SEQ ID NO: 13. In yet other embodiments, the canine hinge region comprises an amino acid sequence that has at least 85% identity to SEQ ID NO: 13. In still other embodiments, the canine hinge region comprises an amino acid sequence that has at least 90% identity to SEQ ID NO: 13. In yet other embodiments, the canine hinge region comprises an amino acid sequence that has at least 95% identity to SEQ ID NO: 13. In specific embodiments, the canine hinge region comprises the amino acid sequence of SEQ ID NO: 13. In other embodiments, the canine hinge region comprises an amino acid sequence that has at least 80% identity to SEQ ID NO: 14. In yet other embodiments, the canine hinge region comprises an amino acid sequence that has at least 85% identity to SEQ ID NO: 14. In still other embodiments, the canine hinge region comprises an amino acid sequence that has at least 90% identity to SEQ ID NO: 14. In yet other embodiments, the canine hinge region comprises an amino acid sequence that has at least 95% identity to SEQ ID NO: 14. In specific embodiments, the canine hinge region comprises the amino acid sequence of SEQ ID NO: 14. In alternative embodiments, the canine hinge region comprises an amino acid sequence that has at least 80% identity to SEQ ID NO: 15. In yet other embodiments, the canine hinge region comprises an amino acid sequence that has at least 85% identity to SEQ ID NO: 15. In still other embodiments, the canine hinge region comprises an amino acid sequence that has at least 90% identity to SEQ ID NO: 15. In yet other embodiments, the canine hinge region comprises an amino acid sequence that has at least 95% identity to SEQ ID NO: 15. In specific embodiments, the canine hinge region comprises the amino acid sequence of SEQ ID NO: 15. In other embodiments, the canine hinge region comprises an amino acid sequence that has at least 80% identity to SEQ ID NO: 16. In yet other embodiments, the canine hinge region comprises an amino acid sequence that has at least 85% identity to SEQ ID NO: 16. In still other embodiments, the canine hinge region comprises an amino acid sequence that has at least 90% identity to SEQ ID NO: 16. In yet other embodiments, the canine hinge region comprises an amino acid sequence that has at least 95% identity to SEQ ID NO: 16. In specific embodiments, the canine hinge region comprises the amino acid sequence of SEQ ID NO: 16.

In particular embodiments, the IgG Fc fusion protein is a canine interleukin-13 receptor alpha 1-canine IgG fusion protein (canine IL-13Rα1-canine IgG fusion protein) that comprises the amino acid sequence of SEQ ID NO: 17. In other embodiments, the canine IL-13Rα1-canine IgG fusion protein comprises an amino acid sequence that has at least 90% identity to the amino acid sequence SEQ ID NO: 17. In yet other embodiments, the canine IL-13Rα1-canine IgG fusion protein comprises an amino acid sequence that has at least 95% identity to the amino acid sequence SEQ ID NO: 17. In still other embodiments, the canine IL-13Rα1-canine IgG fusion protein comprises an amino acid sequence that has at least 97% identity to the amino acid sequence SEQ ID NO: 17. In yet other embodiments, the canine IL-13Rα1-canine IgG fusion protein comprises an amino acid sequence that has at least 98% identity to the amino acid sequence SEQ ID NO: 17. In still other embodiments, the canine IL-13Rα1-canine IgG fusion protein comprises an amino acid sequence that has at least 99% identity to the amino acid sequence SEQ ID NO: 17.

In other particular embodiments, the IgG Fc fusion protein is a canine interleukin-13 receptor alpha 2-canine IgG fusion protein (canine IL-13Rα2-canine IgG fusion protein) that comprises the amino acid sequence of SEQ ID NO: 18. In other embodiments, the canine IL-13Rα2-canine IgG fusion protein comprises an amino acid sequence that has at least 90% identity to the amino acid sequence SEQ ID NO: 18. In yet other embodiments, the canine IL-13Rα2-canine IgG fusion protein comprises an amino acid sequence that has at least 95% identity to the amino acid sequence SEQ ID NO: 18. In still other embodiments, the canine IL-13Rα2-canine IgG fusion protein comprises an amino acid sequence that has at least 97% identity to the amino acid sequence SEQ ID NO: 18. In yet other embodiments, the canine IL-13Rα2-canine IgG fusion protein comprises an amino acid sequence that has at least 98% identity to the amino acid sequence SEQ ID NO: 18. In still other embodiments, the canine IL-13Rα2-canine IgG fusion protein comprises an amino acid sequence that has at least 99% identity to the amino acid sequence SEQ ID NO: 18.

In yet other particular embodiments, the IgG Fc fusion protein is a canine IL-13Rα2-canine IgG fusion protein, that comprises the amino acid sequence of SEQ ID NO: 19. In other embodiments, the canine IL-13Rα2-canine IgG fusion protein comprises an amino acid sequence that has at least 90% identity to the amino acid sequence SEQ ID NO: 19. In yet other embodiments, the canine IL-13Rα2-canine IgG fusion protein comprises an amino acid sequence that has at least 95% identity to the amino acid sequence SEQ ID NO: 19. In still other embodiments, the canine IL-13Rα2-canine IgG fusion protein comprises an amino acid sequence that has at least 97% identity to the amino acid sequence SEQ ID NO: 19. In yet other embodiments, the canine IL-13Rα2-canine IgG fusion protein comprises an amino acid sequence that has at least 98% identity to the amino acid sequence SEQ ID NO: 19. In still other embodiments, the canine IL-13Rα2-canine IgG fusion protein comprises an amino acid sequence that has at least 99% identity to the amino acid sequence SEQ ID NO: 19.

In still other particular embodiments, the IgG Fc fusion protein is a canine IL-13Rα2-canine IgG fusion protein that comprises the amino acid sequence of SEQ ID NO: 20. In other embodiments, the canine IL-13Rα2-canine IgG fusion protein comprises an amino acid sequence that has at least 90% identity to the amino acid sequence SEQ ID NO: 20. In yet other embodiments, the canine IL-13Rα2-canine IgG fusion protein comprises an amino acid sequence that has at least 95% identity to the amino acid sequence SEQ ID NO: 20. In still other embodiments, the canine IL-13Rα2-canine IgG fusion protein comprises an amino acid sequence that has at least 97% identity to the amino acid sequence SEQ ID NO: 20. In yet other embodiments, the canine IL-13Rα2-canine IgG fusion protein comprises an amino acid sequence that has at least 98% identity to the amino acid sequence SEQ ID NO: 20. In still other embodiments, the canine IL-13Rα2-canine IgG fusion protein comprises an amino acid sequence that has at least 99% identity to the amino acid sequence SEQ ID NO: 20.

In yet other particular embodiments, the IgG Fc fusion protein is a canine IL-13Rα2-canine IgG fusion protein that comprises the amino acid sequence of SEQ ID NO: 21. In other embodiments, the canine IL-13Rα2-canine IgG fusion protein comprises an amino acid sequence that has at least 90% identity to the amino acid sequence SEQ ID NO: 21. In yet other embodiments, the canine IL-13Rα2-canine IgG fusion protein comprises an amino acid sequence that has at least 95% identity to the amino acid sequence SEQ ID NO: 21. In still other embodiments, the canine IL-13Rα2-canine IgG fusion protein comprises an amino acid sequence that has at least 97% identity to the amino acid sequence SEQ ID NO: 21. In yet other embodiments, the canine IL-13Rα2-canine IgG fusion protein comprises an amino acid sequence that has at least 98% identity to the amino acid sequence SEQ ID NO: 21. In still other embodiments, the canine IL-13Rα2-canine IgG fusion protein comprises an amino acid sequence that has at least 99% identity to the amino acid sequence SEQ ID NO: 21.

In still other particular embodiments, the IgG Fc fusion protein is a canine IL-13Rα2-canine IgG fusion protein that comprises the amino acid sequence of SEQ ID NO: 22. In other embodiments, the canine IL-13Rα2-canine IgG fusion protein comprises an amino acid sequence that has at least 90% identity to the amino acid sequence SEQ ID NO: 22. In yet other embodiments, the canine IL-13Rα2-canine IgG fusion protein comprises an amino acid sequence that has at least 95% identity to the amino acid sequence SEQ ID NO: 22. In still other embodiments, the canine IL-13Rα2-canine IgG fusion protein comprises an amino acid sequence that has at least 97% identity to the amino acid sequence SEQ ID NO: 22. In yet other embodiments, the canine IL-13Rα2-canine IgG fusion protein comprises an amino acid sequence that has at least 98% identity to the amino acid sequence SEQ ID NO: 22. In still other embodiments, the canine IL-13Rα2-canine IgG fusion protein comprises an amino acid sequence that has at least 99% identity to the amino acid sequence SEQ ID NO: 22.

In yet other particular embodiments, the IgG Fc fusion protein is a canine IL-13Rα2-canine IgG fusion protein that comprises the amino acid sequence of SEQ ID NO: 23. In other embodiments, the canine IL-13Rα2-canine IgG fusion protein comprises an amino acid sequence that has at least 90% identity to the amino acid sequence SEQ ID NO: 23. In yet other embodiments, the canine IL-13Rα2-canine IgG fusion protein comprises an amino acid sequence that has at least 95% identity to the amino acid sequence SEQ ID NO: 23. In still other embodiments, the canine IL-13Rα2-canine IgG fusion protein comprises an amino acid sequence that has at least 97% identity to the amino acid sequence SEQ ID NO: 23. In yet other embodiments, the canine IL-13Rα2-canine IgG fusion protein comprises an amino acid sequence that has at least 98% identity to the amino acid sequence SEQ ID NO: 23. In still other embodiments, the canine IL-13Rα2-canine IgG fusion protein comprises an amino acid sequence that has at least 99% identity to the amino acid sequence SEQ ID NO: 23.

In still other particular embodiments, the IgG Fc fusion protein is a canine IL-13Rα2-canine IgG fusion protein that comprises the amino acid sequence of SEQ ID NO: 24. In other embodiments, the canine IL-13Rα2-canine IgG fusion protein comprises an amino acid sequence that has at least 90% identity to the amino acid sequence SEQ ID NO: 24. In yet other embodiments, the canine IL-13Rα2-canine IgG fusion protein comprises an amino acid sequence that has at least 95% identity to the amino acid sequence SEQ ID NO: 24. In still other embodiments, the canine IL-13Rα2-canine IgG fusion protein comprises an amino acid sequence that has at least 97% identity to the amino acid sequence SEQ ID NO: 24. In yet other embodiments, the canine IL-13Rα2-canine IgG fusion protein comprises an amino acid sequence that has at least 98% identity to the amino acid sequence SEQ ID NO: 24. In still other embodiments, the canine IL-13Rα2-canine IgG fusion protein comprises an amino acid sequence that has at least 99% identity to the amino acid sequence SEQ ID NO: 24.

In yet other particular embodiments, the IgG Fc fusion protein is a canine IL-13Rα2-canine IgG fusion protein that comprises the amino acid sequence of SEQ ID NO: 25. In other embodiments, the canine IL-13Rα2-canine IgG fusion protein comprises an amino acid sequence that has at least 90% identity to the amino acid sequence SEQ ID NO: 25. In yet other embodiments, the canine IL-13Rα2-canine IgG fusion protein comprises an amino acid sequence that has at least 95% identity to the amino acid sequence SEQ ID NO: 25. In still other embodiments, the canine IL-13Rα2-canine IgG fusion protein comprises an amino acid sequence that has at least 97% identity to the amino acid sequence SEQ ID NO: 25. In yet other embodiments, the canine IL-13Rα2-canine IgG fusion protein comprises an amino acid sequence that has at least 98% identity to the amino acid sequence SEQ ID NO: 25. In still other embodiments, the canine IL-13Rα2-canine IgG fusion protein comprises an amino acid sequence that has at least 99% identity to the amino acid sequence SEQ ID NO: 25.

In still other particular embodiments, the IgG Fc fusion protein is a canine IL-13Rα2-canine IgG fusion protein that comprises the amino acid sequence of SEQ ID NO: 26. In other embodiments, the canine TL-13Rα2-canine IgG fusion protein comprises an amino acid sequence that has at least 90% identity to the amino acid sequence SEQ ID NO: 26. In yet other embodiments, the canine IL-13Rα2-canine IgG fusion protein comprises an amino acid sequence that has at least 95% identity to the amino acid sequence SEQ ID NO: 26. In still other embodiments, the canine IL-13Rα2-canine IgG fusion protein comprises an amino acid sequence that has at least 97% identity to the amino acid sequence SEQ ID NO: 26. In yet other embodiments, the canine IL-13Rα2-canine IgG fusion protein comprises an amino acid sequence that has at least 98% identity to the amino acid sequence SEQ ID NO: 26. In still other embodiments, the canine IL-13Rα2-canine IgG fusion protein comprises an amino acid sequence that has at least 99% identity to the amino acid sequence SEQ ID NO: 26.

In yet other particular embodiments, the IgG Fc fusion protein is a canine IL-13Rα2-canine IgG fusion protein that comprises the amino acid sequence of SEQ ID NO: 27. In other embodiments, the canine IL-13Rα2-canine IgG fusion protein comprises an amino acid sequence that has at least 90% identity to the amino acid sequence SEQ ID NO: 27. In yet other embodiments, the canine IL-13Rα2-canine IgG fusion protein comprises an amino acid sequence that has at least 95% identity to the amino acid sequence SEQ ID NO: 27. In still other embodiments, the canine IL-13Rα2-canine IgG fusion protein comprises an amino acid sequence that has at least 97% identity to the amino acid sequence SEQ ID NO: 27. In yet other embodiments, the canine IL-13Rα2-canine IgG fusion protein comprises an amino acid sequence that has at least 98% identity to the amino acid sequence SEQ ID NO: 27. In still other embodiments, the canine IL-13Rα2-canine IgG fusion protein comprises an amino acid sequence that has at least 99% identity to the amino acid sequence SEQ ID NO: 27.

In still other particular embodiments, the IgG Fe fusion protein is a canine IL-13Rα2-canine IgG fusion protein that comprises the amino acid sequence of SEQ ID NO: 28. In other embodiments, the canine IL-13Rα2-canine IgG fusion protein comprises an amino acid sequence that has at least 90% identity to the amino acid sequence SEQ ID NO: 28. In yet other embodiments, the canine IL-13Rα2-canine IgG fusion protein comprises an amino acid sequence that has at least 95% identity to the amino acid sequence SEQ ID NO: 28. In still other embodiments, the canine IL-13Rα2-canine IgG fusion protein comprises an amino acid sequence that has at least 97% identity to the amino acid sequence SEQ ID NO: 28. In yet other embodiments, the canine IL-13Rα2-canine IgG fusion protein comprises an amino acid sequence that has at least 98% identity to the amino acid sequence SEQ ID NO: 28. In still other embodiments, the canine IL-13Rα2-canine IgG fusion protein comprises an amino acid sequence that has at least 99% identity to the amino acid sequence SEQ ID NO: 28.

In yet other particular embodiments, the IgG Fc fusion protein is a canine IL-13Rα2-canine IgG fusion protein that comprises the amino acid sequence of SEQ ID NO: 29. In other embodiments, the canine IL-13Rα2-canine IgG fusion protein comprises an amino acid sequence that has at least 90% identity to the amino acid sequence SEQ ID NO: 29. In yet other embodiments, the canine IL-13Rα2-canine IgG fusion protein comprises an amino acid sequence that has at least 95% identity to the amino acid sequence SEQ ID NO: 29. In still other embodiments, the canine IL-13Rα2-canine IgG fusion protein comprises an amino acid sequence that has at least 97% identity to the amino acid sequence SEQ ID NO: 29. In yet other embodiments, the canine IL-13Rα2-canine IgG fusion protein comprises an amino acid sequence that has at least 98% identity to the amino acid sequence SEQ ID NO: 29. In still other embodiments, the canine IL-13Rα2-canine IgG fusion protein comprises an amino acid sequence that has at least 99% identity to the amino acid sequence SEQ ID NO: 29.

In still other particular embodiments, the IgG Fc fusion protein is a canine IL-13Rα2-canine IgG fusion protein that comprises the amino acid sequence of SEQ ID NO: 30. In other embodiments, the canine IL-13Rα2-canine IgG fusion protein comprises an amino acid sequence that has at least 90% identity to the amino acid sequence SEQ ID NO: 30. In yet other embodiments, the canine IL-13Rα2-canine IgG fusion protein comprises an amino acid sequence that has at least 95% identity to the amino acid sequence SEQ ID NO: 30. In still other embodiments, the canine IL-13Rα2-canine IgG fusion protein comprises an amino acid sequence that has at least 97% identity to the amino acid sequence SEQ ID NO: 30. In yet other embodiments, the canine IL-13Rα2-canine IgG fusion protein comprises an amino acid sequence that has at least 98% identity to the amino acid sequence SEQ ID NO: 30. In still other embodiments, the canine IL-13Rα2-canine IgG fusion protein comprises an amino acid sequence that has at least 99% identity to the amino acid sequence SEQ ID NO: 30.

In yet other particular embodiments, the IgG Fc fusion protein is a canine IL-13Rα2-canine IgG fusion protein that comprises the amino acid sequence of SEQ ID NO: 31. In other embodiments, the canine IL-13Rα2-canine IgG fusion protein comprises an amino acid sequence that has at least 90% identity to the amino acid sequence SEQ ID NO: 31. In yet other embodiments, the canine IL-13Rα2-canine IgG fusion protein comprises an amino acid sequence that has at least 95% identity to the amino acid sequence SEQ ID NO: 31. In still other embodiments, the canine IL-13Rα2-canine IgG fusion protein comprises an amino acid sequence that has at least 97% identity to the amino acid sequence SEQ ID NO: 31. In yet other embodiments, the canine IL-13Rα2-canine IgG fusion protein comprises an amino acid sequence that has at least 98% identity to the amino acid sequence SEQ ID NO: 31. In still other embodiments, the canine IL-13Rα2-canine IgG fusion protein comprises an amino acid sequence that has at least 99% identity to the amino acid sequence SEQ ID NO: 31.

In still other particular embodiments, the IgG Fc fusion protein is a canine IL-13Rα2-canine IgG fusion protein that comprises the amino acid sequence of SEQ ID NO: 32. In other embodiments, the canine IL-13Rα2-canine IgG fusion protein comprises an amino acid sequence that has at least 90% identity to the amino acid sequence SEQ ID NO: 32. In yet other embodiments, the canine IL-13Rα2-canine IgG fusion protein comprises an amino acid sequence that has at least 95% identity to the amino acid sequence SEQ ID NO: 32. In still other embodiments, the canine IL-13Rα2-canine IgG fusion protein comprises an amino acid sequence that has at least 97% identity to the amino acid sequence SEQ ID NO: 32. In yet other embodiments, the canine IL-13Rα2-canine IgG fusion protein comprises an amino acid sequence that has at least 98% identity to the amino acid sequence SEQ ID NO: 32. In still other embodiments, the canine IL-13Rα2-canine IgG fusion protein comprises an amino acid sequence that has at least 99% identity to the amino acid sequence SEQ ID NO: 32.

In yet other particular embodiments, the IgG Fc fusion protein is a canine IL-13Rα2-canine IgG fusion protein that comprises the amino acid sequence of SEQ ID NO: 33. In other embodiments, the canine IL-13Rα2-canine IgG fusion protein comprises an amino acid sequence that has at least 90% identity to the amino acid sequence SEQ ID NO: 33. In yet other embodiments, the canine IL-13Rα2-canine IgG fusion protein comprises an amino acid sequence that has at least 95% identity to the amino acid sequence SEQ ID NO: 33. In still other embodiments, the canine IL-13Rα2-canine IgG fusion protein comprises an amino acid sequence that has at least 97% identity to the amino acid sequence SEQ ID NO: 33. In yet other embodiments, the canine IL-13Rα2-canine IgG fusion protein comprises an amino acid sequence that has at least 98% identity to the amino acid sequence SEQ ID NO: 33. In still other embodiments, the canine IL-13Rα2-canine IgG fusion protein comprises an amino acid sequence that has at least 99% identity to the amino acid sequence SEQ ID NO: 33.

In still other particular embodiments, the IgG Fc fusion protein is a canine IL-13Rα2-canine IgG fusion protein that comprises the amino acid sequence of SEQ ID NO: 34. In other embodiments, the canine TL-13Rα2-canine IgG fusion protein comprises an amino acid sequence that has at least 90% identity to the amino acid sequence SEQ ID NO: 34. In yet other embodiments, the canine IL-13Rα2-canine IgG fusion protein comprises an amino acid sequence that has at least 95% identity to the amino acid sequence SEQ ID NO: 34. In still other embodiments, the canine IL-13Rα2-canine IgG fusion protein comprises an amino acid sequence that has at least 97% identity to the amino acid sequence SEQ ID NO: 34. In yet other embodiments, the canine IL-13Rα2-canine IgG fusion protein comprises an amino acid sequence that has at least 98% identity to the amino acid sequence SEQ ID NO: 34. In still other embodiments, the canine IL-13Rα2-canine IgG fusion protein comprises an amino acid sequence that has at least 99% identity to the amino acid sequence SEQ ID NO: 34.

In yet other particular embodiments, the IgG Fc fusion protein is a canine IL-13Rα2-canine IgG fusion protein that comprises the amino acid sequence of SEQ ID NO: 35. In other embodiments, the canine IL-13Rα2-canine IgG fusion protein comprises an amino acid sequence that has at least 90% identity to the amino acid sequence SEQ ID NO: 35. In yet other embodiments, the canine IL-13Rα2-canine IgG fusion protein comprises an amino acid sequence that has at least 95% identity to the amino acid sequence SEQ ID NO: 35. In still other embodiments, the canine IL-13Rα2-canine IgG fusion protein comprises an amino acid sequence that has at least 97% identity to the amino acid sequence SEQ ID NO: 35. In yet other embodiments, the canine IL-13Rα2-canine IgG fusion protein comprises an amino acid sequence that has at least 98% identity to the amino acid sequence SEQ ID NO: 35. In still other embodiments, the canine IL-13Rα2-canine IgG fusion protein comprises an amino acid sequence that has at least 99% identity to the amino acid sequence SEQ ID NO: 35.

As indicated below, unless otherwise specifically indicated, an antibody of the present invention is an IgG antibody. In specific embodiments, the antibody of the present invention is a feline antibody. In related embodiments, the antibody is a felinized antibody. In other embodiments, the antibody is a canine antibody. In still other embodiments, the antibody is a caninized antibody.

In particular embodiments, the caninized antibody is a caninized canine interleukin-31 receptor alpha (cIL-31RA) antibody. In more specific embodiments, the heavy chain of the cIL-31RA caninized antibody comprises a variable region comprising the amino acid sequence of SEQ ID NO: 44. In more specific embodiments, the antibody further comprises a light chain of the cIL-31 RA caninized antibody. In particular embodiments, the light chain of the cIL-31RA caninized antibody comprises the amino acid sequence of SEQ ID NO: 45. In other embodiments, the light chain of the cIL-31RA caninized antibody comprises an amino acid sequence that has at least 90% identity to the amino acid sequence SEQ ID NO: 45. In yet other embodiments, the light chain of the cIL-3 IRA caninized antibody comprises an amino acid sequence that has at least 95% identity to the amino acid sequence SEQ ID NO: 45. In still other embodiments, the light chain of the cIL-3 IRA caninized antibody comprises an amino acid sequence that has at least 97% identity to the amino acid sequence SEQ ID NO: 45. In yet other embodiments, the light chain of the cIL-3 IRA caninized antibody comprises an amino acid sequence that has at least 98% identity to the amino acid sequence SEQ ID NO: 45. In still other embodiments, the light chain of the cIL-31RA caninized antibody comprises an amino acid sequence that has at least 99% identity to the amino acid sequence SEQ ID NO: 45.

In particular embodiments, the heavy chain of the cIL-31RA caninized antibody comprises the amino acid sequence of SEQ ID NO: 36. In other embodiments, the heavy chain of the cIL-31RA caninized antibody comprises an amino acid sequence that has at least 90% identity to the amino acid sequence SEQ ID NO: 36. In yet other embodiments, the heavy chain of the cIL-31RA caninized antibody comprises an amino acid sequence that has at least 95% identity to the amino acid sequence SEQ ID NO: 36. In still other embodiments, the heavy chain of the cIL-31RA caninized antibody comprises an amino acid sequence that has at least 97% identity to the amino acid sequence SEQ ID NO: 36. In yet other embodiments, the heavy chain of the cIL-31RA caninized antibody comprises an amino acid sequence that has at least 98% identity to the amino acid sequence SEQ ID NO: 36. In still other embodiments, the heavy chain of the cIL-31RA caninized antibody comprises an amino acid sequence that has at least 99% identity to the amino acid sequence SEQ ID NO: 36.

In yet other particular embodiments, the heavy chain of the cIL-31RA caninized antibody comprises the amino acid sequence of SEQ ID NO: 37. In still other embodiments, the heavy chain of the cIL-31RA caninized antibody comprises an amino acid sequence that has at least 90% identity to the amino acid sequence SEQ ID NO: 37. In yet other embodiments, the heavy chain of the cIL-31RA caninized antibody comprises an amino acid sequence that has at least 95% identity to the amino acid sequence SEQ ID NO: 37. In still other embodiments, the heavy chain of the cIL-31RA caninized antibody comprises an amino acid sequence that has at least 97% identity to the amino acid sequence SEQ ID NO: 37. In yet other embodiments, the heavy chain of the cIL-31RA caninized antibody comprises an amino acid sequence that has at least 98% identity to the amino acid sequence SEQ ID NO: 37. In still other embodiments, the heavy chain of the cIL-31RA caninized antibody comprises an amino acid sequence that has at least 99% identity to the amino acid sequence SEQ ID NO: 37.

In yet other particular embodiments, the heavy chain of the cIL-31RA caninized antibody comprises the amino acid sequence of SEQ ID NO: 38. In still other embodiments, the heavy chain of the cIL-31RA caninized antibody comprises an amino acid sequence that has at least 90% identity to the amino acid sequence SEQ ID NO: 38. In yet other embodiments, the heavy chain of the cIL-31RA caninized antibody comprises an amino acid sequence that has at least 95% identity to the amino acid sequence SEQ ID NO: 38. In still other embodiments, the heavy chain of the cIL-31RA caninized antibody comprises an amino acid sequence that has at least 97% identity to the amino acid sequence SEQ ID NO: 38. In yet other embodiments, the heavy chain of the cIL-31RA caninized antibody comprises an amino acid sequence that has at least 98% identity to the amino acid sequence SEQ ID NO: 38. In still other embodiments, the heavy chain of the cIL-31RA caninized antibody comprises an amino acid sequence that has at least 99% identity to the amino acid sequence SEQ ID NO: 38.

In yet other particular embodiments, the heavy chain of the cIL-31RA caninized antibody comprises the amino acid sequence of SEQ ID NO: 39. In still other embodiments, the heavy chain of the cIL-31RA caninized antibody comprises an amino acid sequence that has at least 90% identity to the amino acid sequence SEQ ID NO: 39. In yet other embodiments, the heavy chain of the cIL-31RA caninized antibody comprises an amino acid sequence that has at least 95% identity to the amino acid sequence SEQ ID NO: 39. In still other embodiments, the heavy chain of the cIL-31RA caninized antibody comprises an amino acid sequence that has at least 97% identity to the amino acid sequence SEQ ID NO: 39. In yet other embodiments, the heavy chain of the cIL-31RA caninized antibody comprises an amino acid sequence that has at least 98% identity to the amino acid sequence SEQ ID NO: 39. In still other embodiments, the heavy chain of the cIL-31RA caninized antibody comprises an amino acid sequence that has at least 99% identity to the amino acid sequence SEQ ID NO: 39.

In yet other particular embodiments, the heavy chain of the cIL-31RA caninized antibody comprises the amino acid sequence of SEQ ID NO: 40. In still other embodiments, the heavy chain of the cIL-31RA caninized antibody comprises an amino acid sequence that has at least 90% identity to the amino acid sequence SEQ ID NO: 40. In yet other embodiments, the heavy chain of the cIL-31RA caninized antibody comprises an amino acid sequence that has at least 95% identity to the amino acid sequence SEQ ID NO: 40. In still other embodiments, the heavy chain of the cIL-31RA caninized antibody comprises an amino acid sequence that has at least 97% identity to the amino acid sequence SEQ ID NO: 40. In yet other embodiments, the heavy chain of the cIL-31RA caninized antibody comprises an amino acid sequence that has at least 98% identity to the amino acid sequence SEQ ID NO: 40. In still other embodiments, the heavy chain of the cIL-31RA caninized antibody comprises an amino acid sequence that has at least 99% identity to the amino acid sequence SEQ ID NO: 40.

In yet other particular embodiments, the heavy chain of the cIL-31RA caninized antibody comprises the amino acid sequence of SEQ ID NO: 41. In still other embodiments, the heavy chain of the cIL-31RA caninized antibody comprises an amino acid sequence that has at least 90% identity to the amino acid sequence SEQ ID NO: 41. In yet other embodiments, the heavy chain of the cIL-31RA caninized antibody comprises an amino acid sequence that has at least 95% identity to the amino acid sequence SEQ ID NO: 41. In still other embodiments, the heavy chain of the cIL-31RA caninized antibody comprises an amino acid sequence that has at least 97% identity to the amino acid sequence SEQ ID NO: 41. In yet other embodiments, the heavy chain of the cIL-31RA caninized antibody comprises an amino acid sequence that has at least 98% identity to the amino acid sequence SEQ ID NO: 41. In still other embodiments, the heavy chain of the cIL-31RA caninized antibody comprises an amino acid sequence that has at least 99% identity to the amino acid sequence SEQ ID NO: 41.

In yet other particular embodiments, the heavy chain of the cIL-31RA caninized antibody comprises the amino acid sequence of SEQ ID NO: 42. In still other embodiments, the heavy chain of the cIL-31RA caninized antibody comprises an amino acid sequence that has at least 90% identity to the amino acid sequence SEQ ID NO: 42. In yet other embodiments, the heavy chain of the cIL-31RA caninized antibody comprises an amino acid sequence that has at least 95% identity to the amino acid sequence SEQ ID NO: 42. In still other embodiments, the heavy chain of the cIL-31RA caninized antibody comprises an amino acid sequence that has at least 97% identity to the amino acid sequence SEQ ID NO: 42. In yet other embodiments, the heavy chain of the cIL-31RA caninized antibody comprises an amino acid sequence that has at least 98% identity to the amino acid sequence SEQ ID NO: 42. In still other embodiments, the heavy chain of the cIL-31RA caninized antibody comprises an amino acid sequence that has at least 99% identity to the amino acid sequence SEQ ID NO: 42.

In yet other particular embodiments, the heavy chain of the cIL-31RA caninized antibody comprises the amino acid sequence of SEQ ID NO: 43. In still other embodiments, the heavy chain of the cIL-31RA caninized antibody comprises an amino acid sequence that has at least 90% identity to the amino acid sequence SEQ ID NO: 43. In yet other embodiments, the heavy chain of the cIL-31RA caninized antibody comprises an amino acid sequence that has at least 95% identity to the amino acid sequence SEQ ID NO: 43. In still other embodiments, the heavy chain of the cIL-31RA caninized antibody comprises an amino acid sequence that has at least 97% identity to the amino acid sequence SEQ ID NO: 43. In yet other embodiments, the heavy chain of the cIL-31RA caninized antibody comprises an amino acid sequence that has at least 98% identity to the amino acid sequence SEQ ID NO: 43. In still other embodiments, the heavy chain of the cIL-31RA caninized antibody comprises an amino acid sequence that has at least 99% identity to the amino acid sequence SEQ ID NO: 43.

The present invention further provides individual nucleic acids comprising individual nucleotide sequences that encode the individual antibody heavy chains, antibody light chains, or IgG fusion proteins of the invention. Accordingly, the present invention provides a nucleic acid comprising a nucleotide sequence that encodes an antibody heavy chain, an antibody light chain, or an IgG fusion protein of the invention. In particular embodiments, the present invention provides nucleic acids comprising a nucleotide sequence that encodes any one of the canine-13Rα1-canine IgG fusion proteins of the invention. In related particular embodiments, the present invention provides nucleic acids comprising a nucleotide sequence that encodes any one of the canine-13Rα2-canine IgG fusion proteins of the invention. In specific embodiments, the present invention provides nucleic acids comprising a nucleotide sequence that encodes the heavy chain of the cIL-31RA caninized antibody that comprises a variable region comprising the amino acid sequence of SEQ ID NO: 44. In related embodiments, the present invention provides nucleic acids comprising a nucleotide sequence that encodes any one of the heavy chains of a cIL-31RA caninized antibody. The present invention also provides nucleic acids comprising a nucleotide sequence that encodes any one of the light chains of the cIL-31RA caninized antibody. In addition, the present invention provides nucleic acids comprising multiple nucleotide sequences that each encode an antibody heavy and a light chain of the present invention. The present invention further provides nucleic acids comprising multiple nucleotide sequences that each encode an IgG fusion protein of the invention. The present invention further provides vectors that comprise these nucleic acids. In particular embodiments, the vector is an expression vector. The present invention further provides host cells that comprise any of the vectors of the present invention.

The present invention also provides pharmaceutical compositions comprising antibodies and IgG Fc fusion proteins with enhanced half-lives of the invention and a pharmaceutically acceptable carrier. This enhanced half-life is due to, at least in part, the antibodies and IgG Fc fusion proteins comprising a fragment crystallizable region (Fc) that comprises one or more amino acid substitutions in their Fc regions. In more specific embodiments, the pharmaceutical comprises a canine-13Rα1-canine IgG fusion protein of the invention and a pharmaceutically acceptable carrier. In related embodiments, the pharmaceutical comprises a canine-13Rα2-canine IgG fusion protein of the invention and a pharmaceutically acceptable carrier. In yet another embodiment the pharmaceutical comprises a cIL-31RA caninized antibody of the invention and a pharmaceutically acceptable carrier. The present invention also provides pharmaceutical compositions that comprise a combination of a canine-13Rα1-canine IgG fusion protein, a canine-13Rα2-canine IgG fusion protein, and/or a caninized cIL-31RA caninized antibody. The present invention further provides methods of treating a canine that has atopic dermatitis comprising administering to the canine any one or more of the pharmaceutical compositions of the present invention.

These and other aspects of the present invention will be better appreciated by reference to the Detailed Description and FIGURE.

### BRIEF DESCRIPTION OF THE DRAWINGS

FIG. 1 depicts an alignment of the amino acid sequences of the Fc regions from human IgG1 Fc [SEQ ID NO: 54], canine IgG-A [SEQ ID NO: 1], canine IgG-B [SEQ ID NO: 3], canine IgG-C [SEQ ID NO: 5], canine IgG-D [SEQ ID NO: 7], feline IgG-1a [SEQ ID NO: 52], and feline IgG-2 [SEQ ID NO: 53], along with a consensus sequence [SEQ ID NO: 55]. The feline IgG-1a and feline IgG-2 Fc regions depicted have two additional N-terminal amino acid residues relative to the corresponding feline Fc regions defined by Strietzel et al., [Vet Immunol & Immunopathol., 158:214-223 (2014)]. The solid arrows denote particular amino acid residue positions that reflect the EU numbering scheme according to Sequences of Proteins of Immunological Interest, 5th ed., Kabat et al., National Institutes of Health, Bethesda, Md. (1991). The “x” reflects the lack of uniformity at position 252 in the consensus sequence for the seven amino acid sequences being compared.

### DETAILED DESCRIPTION OF THE INVENTION

Binding of antibodies and/or IgG Fc fusion proteins to FcRn is highly dependent on pH. Accordingly, the binding occurs with high affinity at moderately acidic pH in the endosomal compartments, but with a significant lower binding affinity at physiological pH at the cell surface. Whereas the strong binding of IgG antibodies to FcRn in the endosomal compartment both protects the antibody from the degradative action of proteolytic enzymes in endosomes and allows the recycling of the receptor-bound antibody at the cell surface, the higher pH at the cell surface (i.e., physiological pH), weakens that binding and thereby, allows the release of the antibody into the circulation. Substituting one or more amino acids in the Fc region can serve to increase the binding affinity of antibodies and/or IgG Fc fusion proteins to FcRn at moderately acidic pH, and thereby increase the half-life of the antibodies and/or IgG Fc fusion proteins in vivo.

The present invention provides antibodies and IgG Fc fusion proteins that comprise a fragment crystallizable region (Fc region) comprising one or more amino acid specific substitutions in their Fc regions. In one aspect of the present invention, these antibodies and IgG Fc fusion proteins have an enhanced binding affinity for their neonatal Fc receptor (FcRn) at moderately acidic pH (pH 5.5-pH 6.5) and extend the half-life of the antibodies and/or IgG Fc fusion proteins relative to the corresponding antibodies and/or IgG Fc fusion proteins with unsubstituted Fc regions. In related embodiments, the antibodies and/or IgG Fc fusion proteins have an enhanced binding affinity for their FcRn at moderately acidic pH, but an unmodified or minimally modified binding affinity for their FcRn at physiological pH (pH 7.2-7.6). Therefore, in one aspect of the present invention, the specific amino acid substitutions to the Fc regions of the antibodies and/or IgG Fc fusion proteins do not appreciably affect the release of the antibody and/or IgG Fc fusion protein from their FcRn at physiological pH, but do significantly increase the binding affinity of the antibodies and/or IgG Fc fusion proteins for their FcRn at moderately acidic pH.

Alternatively, or in conjunction, increasing the differential between the binding affinity at moderately acidic pH and the binding affinity at physiological pH of antibodies and/or IgG Fc fusion proteins to FcRn, can also extend the relative in vivo half-life of the antibodies and/or IgG Fc fusion proteins. The present invention therefore further provides antibodies and IgG Fc fusion proteins that comprise a fragment crystallizable region (Fc) that comprises one or more amino acid substitutions in their Fc regions that leads to a larger differential between the binding affinity of antibodies and/or IgG Fc fusion proteins for their neonatal Fc receptor (FcRn) at moderately acidic pH than their binding affinity at the physiological pH relative to that differential between those corresponding antibodies and IgG Fc fusion proteins that do not comprise amino acid substitutions in their Fc regions. In particular embodiments of this type the antibodies and IgG Fc fusion proteins also have an enhanced binding affinity for their neonatal Fc receptor (FcRn) at moderately acidic pH.

### ABBREVIATIONS

Throughout the detailed description and examples of the invention the following abbreviations will be used:
ADCC Antibody-dependent cellular cytotoxicity
CDC Complement-dependent cytotoxicity
CDR Complementarity determining region in the immunoglobulin variable regions, defined using the Kabat numbering system
cFc Canine fragment crystallizable region
CHO Chinese hamster ovary
EC50 concentration resulting in 50% efficacy or binding
ECD Extracellular domain
ELISA Enzyme-linked immunosorbant assay
EU index The widely used numbering according to: Sequences of Proteins of Immunological Interest, 5th ed., Kabat et al., National Institutes of Health, Bethesda, Md. (1991), also referred to as the EU index, the EU index of Kabat, or the EU numbering scheme.
fFc Feline fragment crystallizable region
FR Antibody framework region: the immunoglobulin variable regions excluding the CDR regions.
IC50 concentration resulting in 50% inhibition
IgG Immunoglobulin G
Kabat An immunoglobulin alignment and numbering system pioneered by Elvin A. Kabat [Sequences of Proteins of Immunological Interest, 5th Ed. Public Health Service, National Institutes of Health, Bethesda, Md. (1991)]
mAb Monoclonal antibody (also Mab or MAb)
PCR Polymerase chain reaction
PK Pharmacokinetics
V region The segment of IgG chains which is variable in sequence between different antibodies.
VH Immunoglobulin heavy chain variable region
VL Immunoglobulin light chain variable region
Vk Immunoglobulin kappa light chain variable region
VI Immunoglobulin lambda light chain variable region

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Activity” may refer to modulation of components of the innate or the adaptive immune systems.

“Administration” and “treatment”, as it applies to an animal, e.g., a canine or feline subject, cell, tissue, organ, or biological fluid, refers to contact of an exogenous pharmaceutical, therapeutic, diagnostic agent, or composition to the animal e.g., a canine or feline subject, cell, tissue, organ, or biological fluid. Treatment of a cell encompasses contact of a reagent to the cell, as well as contact of a reagent to a fluid, where the fluid is in contact with the cell.

“Administration” and “treatment” also mean in vitro and ex vivo treatments, e.g., of a cell, by a reagent, diagnostic, binding compound, or by another cell. The term “subject” includes any organism, preferably an animal, more preferably a mammal (e.g., canine, feline, or human) and most preferably a canine or feline.

“Treat” or “treating” means to administer a therapeutic agent, such as a composition comprising the IgG fusion protein proteins (e.g., canine or feline IgG fusion proteins) and/or antibodies of the present invention (e.g., caninized, felinized, canine or feline antibodies), internally or externally to e.g., a non-human subject such as a canine or feline, or canine or feline patient having one or more symptoms, or being suspected of having a condition, for which the agent has therapeutic activity. Typically, the therapeutic agent is administered in an amount effective to alleviate and/or ameliorate one or more disease/condition symptoms (e.g., atopic dermatitis or cancer) in the treated subject or population, whether by inducing the regression of or inhibiting the progression of such symptom(s) by any clinically measurable degree. The amount of a therapeutic agent that is effective to alleviate any particular disease/condition symptom (also referred to as the “therapeutically effective amount”) may vary according to factors such as the disease state, age, and weight of the patient (e.g., canine or feline), and the ability of the pharmaceutical composition to elicit a desired response in the subject. Whether a disease/condition symptom has been alleviated or ameliorated can be assessed by any clinical measurement typically used by veterinarians or other skilled healthcare providers to assess the severity or progression status of that symptom. While an embodiment of the present invention (e.g., a treatment method or article of manufacture) may not be effective in alleviating the target disease/condition symptom(s) in every subject, it should alleviate the target disease/condition symptom(s) in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Treatment,” as it applies to a human, veterinary (e.g., canine or feline), or research subject, refers to therapeutic treatment, as well as research and diagnostic applications, as indicated above, and includes contact of the antibodies and/or fusion proteins of the present invention to e.g., a canine, feline, or other animal subject, a cell, a tissue, a physiological compartment, or a physiological fluid.

As used herein “moderately acidic pH” is in the range of pH 5.5-pH 6.5. In the examples below, pH 6.0 was used as the moderately acidic pH.

As used herein “physiological pH” is in the range of pH 7.2-pH 7.6. In the examples below, pH 7.4 was used as the physiological pH.

As used herein, the term “canine” includes all domestic dogs, Canis lupus familiaris or Canis familiaris, unless otherwise indicated.

As used herein, the term “feline” refers to any member of the Felidae family. Members of this family include wild, zoo, and domestic members, including domestic cats, pure-bred and/or mongrel companion cats, show cats, laboratory cats, cloned cats, and wild or feral cats.

As used herein, the term “antibody” refers to any form of antibody that exhibits the desired biological activity. An antibody can be a monomer, dimer, or larger multimer. Thus, it is used in the broadest sense and specifically covers, but is not limited to, monoclonal antibodies (including full length monoclonal antibodies), polyclonal antibodies, multi-specific antibodies (e.g., bispecific antibodies), and camelized single domain antibodies. Unless otherwise specifically indicated, an antibody of the present invention is an IgG antibody.

As used herein, an “IgG antibody” is an immunoglobulin G antibody that comprises two heavy chains and two light chains. IgG antibodies of the present invention include caninized antibodies, fully canine antibodies, felinized antibodies, fully feline antibodies, and corresponding chimeric antibodies.

“Parental antibodies” are antibodies obtained by exposure of an immune system to an antigen prior to modification of the antibodies for an intended use, such as caninization of an antibody for use as a canine therapeutic antibody.

As used herein, an antibody of the present invention that “blocks” or is “blocking” or is “blocking the binding” of e.g., a canine receptor to its binding partner (e.g., its ligand) or a feline receptor to its binding partner (e.g., its ligand), is an antibody that blocks (partially or fully) the binding of the ligand and its receptor and vice versa, as determined in standard binding assays (e.g., BIACore®, ELISA, or flow cytometry).

Typically, an antibody or antigen binding fragment of the invention retains at least 10% of its canine or feline antigen binding activity (when compared to the parental antibody) when that activity is expressed on a molar basis. Preferably, an antibody or antigen binding fragment of the invention retains at least 20%, 50%, 70%, 80%, 90%, 95% or 100% or more of the canine antigen binding affinity as the parental antibody. It is also intended that an antibody or antigen binding fragment of the invention can include conservative or non-conservative amino acid substitutions (referred to as “conservative variants” or “function conserved variants” of the antibody) that do not substantially alter its biologic activity.

An “isolated antibody” refers to the purification status and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

As used herein, a “chimeric antibody” is an antibody having the variable domain from a first antibody and the constant domain from a second antibody, where the first and second antibodies are from different species. [U.S. Pat. No. 4,816,567; and Morrison et al., Proc. Natl. Acad. Sci. USA 81: 6851-6855 (1984)]. Typically the variable domains are obtained from an antibody from an experimental animal (the “parental antibody”), such as a rodent, and the constant domain sequences are obtained from the animal subject antibodies, e.g., canine, feline, or human so that the resulting chimeric antibody will be less likely to elicit an adverse immune response in the canine, feline or human subject respectively, than the parental (e.g., rodent) antibody.

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat, Adv. Prot. Chem. 32:1-75 (1978); Kabat, et al., J. Biol. Chem. 252:6609-6616 (1977)].

As used herein, the term “hypervariable region” refers to the amino acid residues of an antibody that are responsible for antigen-binding. The hypervariable region comprises amino acid residues from a “complementarity determining region” or “CDR” [i.e. CDRL1 (or LCDR1), CDRL2 (or LCDR2), and CDRL3 (or LCDR3) in the light chain variable domain and CDRH1 (or HCDR1), CDRH2 (or HCDR2), and CDRH3 (or HCDR3) in the heavy chain variable domain]. [See Kabat et al. Sequences of Proteins of Immunological Interest, 5th Ed. Public Health Service, National Institutes of Health, Bethesda, Md. (1991), defining the CDR regions of an antibody by sequence; see also Chothia and Lesk, J. Mol. Biol. 196: 901-917 (1987) defining the CDR regions of an antibody by structure].

As used herein, the term “framework” or “FR” residues refers to those variable domain residues other than the hypervariable region residues defined herein as CDR residues.

The “fragment crystallizable region” abbreviated as “Fc” and used interchangeably with “Fe region” corresponds to the CH2-CH3 portion of an antibody that interacts with cell surface receptors called Fc receptors.

As used herein a “wild type Fc” is used interchangeably with a “wild type Fc region” and is an Fc region (e.g., cFc region or fFc region) such as one found in nature that does not comprise any amino acid substitutions that were made to enhance the half-life of an antibody or an IgG Fc fusion protein that comprises the wild type Fc region. As noted below, an Fc region comprising one or more amino acid substitutions made to diminish the antibody-dependent cytotoxicity (ADCC) and complement-dependent cytotoxicity (CDC) of the antibody or IgG fusion protein, such as the canine IgG-Bm region, is not found in nature and therefore, is not a wild type Fc region. However, an antibody or IgG fusion protein comprising such an Fc region (e.g., a canine IgG-Bm Fc region) should have the same half-life as the corresponding antibody or IgG Fc fusion protein that comprises the corresponding wild type Fc region found in nature (i.e., the canine IgG-B Fc region) in the absence of any amino acid substitutions made to enhance the half-life of an antibody or an IgG Fc fusion protein comprising the canine IgG-Bm region.

As used herein the term “canine frame” refers to the amino acid sequence of the heavy chain and light chain of a canine antibody other than the hypervariable region residues defined herein as CDR residues. With regard to a caninized antibody, in the majority of embodiments the amino acid sequences of the native canine CDRs are replaced with the corresponding foreign CDRs (e.g., those from a mouse, rat, or human) in both chains. Optionally the heavy and/or light chains of the canine antibody may contain some foreign non-CDR residues, e.g., so as to preserve the conformation of the foreign CDRs within the canine antibody, and/or to modify the Fc region function, as exemplified below and/or disclosed in U.S. Pat. No. 10,106,607 B2, hereby incorporated by reference herein in its entirety. The present invention provides additional amino acid substitutions to the Fc region of the canine frame to enhance the half-life of the antibodies of the present invention.

As used herein the term “feline frame” refers to the amino acid sequence of the heavy chain and light chain of a feline antibody other than the hypervariable region residues defined herein as CDR residues. With regard to a felinized antibody, in the majority of embodiments the amino acid sequences of the native feline CDRs are replaced with the corresponding foreign CDRs (e.g., those from a mouse, rat, or human) in both chains. Optionally the heavy and/or light chains of the feline antibody may contain some foreign non-CDR residues, e.g., so as to preserve the conformation of the foreign CDRs within the feline antibody, and/or to modify the Fc region function, as exemplified below and/or disclosed for caninized antibodies in U.S. Pat. No. 10,106,607 B2, hereby incorporated by reference herein in its entirety. The present invention provides additional amino acid substitutions to the Fc region of the feline frame to enhance the half-life of the antibodies of the present invention.

As used herein a “canine fragment crystallizable region” is interchangeably abbreviated as “cFc region” or just “cFc” and corresponds to a canine fragment crystallizable region from a canine antibody. The canine fragment crystallizable region (cFc) of each of the four canine IgGs were first described by Tang et al. [Vet. Immunol. Immunopathol. 80: 259-270 (2001); see also, Bergeron et al., Vet. Immunol. Immunopathol. 157: 31-41 (2014)].

As used herein a “feline fragment crystallizable region” is interchangeably abbreviated as “fFc region” or just “fFc” and corresponds to a feline fragment crystallizable region from a feline antibody. The feline fragment crystallizable region (fFc) of each of the three feline IgGs were described by [Strietzel et al., Vet Immunol & Immunopathol. 158:214-223 (2014)].

There are four known IgG heavy chain subtypes of canine IgG and two known light chain subtypes. The four IgG heavy chains are referred to as A, B, C, and D. These heavy chains represent four different subclasses of dog IgG, which are referred to as IgG-A (or IgGA), IgG-B (or IgGB), IgG-C (or IgGC) and IgG-D (or IgGD). Each heavy chain consists of one variable domain (VH) and three constant domains referred to as CH1, CH2, and CH3. The CH1 domain is connected to the CH2 domain via an amino acid sequence referred to as the “hinge” or alternatively as the “hinge region”. The DNA and amino acid sequences of these four heavy chains IgGs were first identified by Tang et al. [Vet. Immunol. Immunopathol. 80:259-270 (2001)]. The amino acid and DNA sequences for these heavy chains IgGs are also available from the GenBank data bases. For example, the amino acid sequence of IgG-A heavy chain has accession number AAL35301.1, IgG-B has accession number AAL35302.1, IgG-C has accession number AAL35303.1, and IgG-D has accession number (AAL35304.1). Canine antibodies also contain two types of light chains, kappa and lambda. The DNA and amino acid sequence of these light chains can be obtained from GenBank Databases. For example, the kappa light chain amino acid sequence has accession number ABY 57289.1 and the lambda light chain has accession number ABY 55569.1.

The constant regions for feline IgG subclasses and their hinge regions have been described [Strietzel et al., Vet Immunol & Immunopathol., 158:214-223 (2014)]. The amino acid sequences of the feline heavy chain constant regions are available from Genbank databases [IgG1a accession number BAA32229.1, IgG1b accession number BAA32230.1 and IgG2 accession number AHH34165.1]. IgG1a and IgG1b appear to be allelic variants, and both bind strongly to human C1q suggesting that they may be able to activate complement. In contrast feline IgG2 has a divergent sequence especially in the hinge region and has negligible binding to C1q.

As used herein, a “substitution of an amino acid residue” with another amino acid residue in an amino acid sequence of an antibody for example, is equivalent to “replacing an amino acid residue” with another amino acid residue and denotes that a particular amino acid residue at a specific position in the amino acid sequence has been replaced by (or substituted for) by a different amino acid residue e.g., by recombinant DNA technology. Such substitutions can be particularly designed i.e., purposefully replacing an asparagine (N) with a phenylalanine (F) at a specific position in the amino acid sequence of an Fc region e.g., at position 434, as numbered according to the EU index as in Kabat. The amino acid substitutions can be made, for example, to increase the half-life of a given antibody or IgG fusion protein, and/or as noted below, to diminish the antibody-dependent cytotoxicity (ADCC) and complement-dependent cytotoxicity (CDC) of the antibody or IgG fusion protein.

An antibody or IgG Fc fusion protein of the invention that comprises a Fc region (e.g., fFc or cFc) that contains one or more amino acid substitutions e.g., at positions 252, 254, 256, 308, 433, 434, 436, or any combination thereof, as numbered according to the EU index as in Kabat, in which that Fc region is identified by its isotype or subtype (e.g., IgG-B), or comprising a specific amino acid sequence in the absence of such substitution, (e.g., a cFc comprising the amino acid sequence of SEQ ID NO: 2) or by having a percent identity to a specific amino acid sequence in the absence of such substitution, (i.e., a cFc comprising 90% identity to the amino acid sequence of SEQ ID NO: 2) as used herein, means that the antibody or IgG Fc fusion protein comprises an IgG-B Fc region, a cFc comprising the amino acid sequence of SEQ ID NO: 2, or a cFc comprising 90% identity to the amino acid sequence of SEQ ID NO: 2, respectively, that specifically contains those one or more amino acid substitutions. Although redundant, this can be reinforced by the statement, wherein the Fc region (e.g., fFc or cFc) “retains said one or more amino acid substitutions”. Accordingly, when a Fc region of the antibody or IgG Fc fusion protein comprises one or more amino acid substitutions is identified as comprising e.g., an IgG-B Fc region, it is understood that the amino acid sequence of the IgG-B Fc region contains those one or more amino acid substitutions. Similarly, when a Fc region of the antibody or IgG Fc fusion protein comprises one or more amino acid substitutions is identified as comprising e.g., the amino acid sequence of SEQ ID NO: 2, it is understood that the Fc region comprises the amino acid sequence of SEQ ID NO: 2 containing those one or more amino acid substitutions. In addition, when a Fc region of the antibody or IgG Fc fusion protein comprises one or more amino acid substitutions is identified as having at least 90% identity to the amino acid sequence of SEQ ID NO: 2, it is understood that the amino acid sequence having at least 90% identity to the amino acid sequence of SEQ ID NO: 2 retains those one or more amino acid substitutions and that the at least 90% identity to the amino acid sequence is in regard to replacements/substitutions at the remaining amino acid positions of the sequence.

As used herein, the canine Fc (cFc) “IgG-Bm” comprises the amino acid sequence of SEQ ID NO: 4, which is the amino acid sequence of canine IgG-B Fc but comprising two (2) amino acid residue substitutions, D31A and N63A, in the amino acid sequence of SEQ ID NO: 3 of IgG-B (see below). Accordingly, both the aspartic acid residue (D) at position 31 of SEQ ID NO: 3 and the asparagine residue (N) at position 63 of SEQ ID NO: 3, are substituted by an alanine residue (A) in the amino acid sequence of IgG-Bm, i.e., SEQ ID NO: 4. These two amino acid residue substitutions serve to significantly diminish the antibody-dependent cytotoxicity (ADCC) and complement-dependent cytotoxicity (CDC) of the naturally occurring canine IgG-B [see, U.S. Pat. No. 10,106,607 B2, the contents of which are hereby incorporated by reference in their entirety]. Analogous substitutions can be made in the IgG-A, IgG-C, and IgG-D Fc regions, which are denoted as IgG-Am, IgG-Cm, and IgG-Dm below. In addition, analogous changes in the amino acid sequences of the feline Fc regions, IgG1a, IgG1b, and IgG2 also are provided in the Examples below and such modifications are denoted as “IgG1am”, “IgG1am”, or “IgG2bm” respectively.

As used herein, the “extracellular domain” or “ECD” of a transmembrane protein, such as canine Interleukin-13 receptor alpha 1, or canine Interleukin-13 receptor alpha 2, refers to the portion of the transmembrane protein that naturally projects into the environment surrounding the cell. The ECD does not include the transmembrane portion of the transmembrane protein. The ECD of canine Interleukin-13 receptor alpha 1 and canine Interleukin-13 receptor alpha 2 both bind to canine IL-13.

As used herein, an “artificial protein” and an “artificial protein molecule” are used interchangeably and denote a protein (or multimer of proteins, such as dimers, heterodimers, tetramers, and heterotetramers, etc.) that does not naturally exist in nature, such as a man-made fusion protein.

As used herein a “fusion protein” is an artificial protein that comprises amino acid sequences from two or more different proteins which are joined together by peptide bonds.

As used herein an “Fc fusion protein” is used interchangeably with the term “IgG Fc fusion protein” and is an artificial protein that joins the Fc region of an IgG antibody, which can further include a hinge region, e.g., the canine IgG-B hinge region-CH2-CH3 or a feline IgG1a hinge region-CH2-CH3, with another biologically active protein domain to generate a molecule with unique structure and therapeutic utility.

As used herein a “cFc fusion protein” is used interchangeably with the term “canine IgG Fc fusion protein” and is an artificial protein that joins the cFc of a canine IgG antibody, which can include a hinge region, e.g., the IgG-B hinge region-CH2-CH3, with another biologically active protein domain to generate a molecule with unique structure and therapeutic utility. For example, a canine IL-13Rα2-cFc fusion protein (cIL-13Rα2-cFc fusion protein) comprises the extracellular domain (ECD) of canine IL-13Rα2 linked to the N-terminus of a canine IgG FFc (cFc). The ECD of the IL-13Rα2 may be linked to the N-terminus of the cFc by a canine hinge region. The cFc fusion proteins of the present invention, although exemplified by the use of the IgGB hinge region and the IgGB cFc, are in no way so limited, but rather they include the corresponding fusion proteins with the cFcs of IgGA, IgGC, and IgGD and optionally the hinge regions of IgGA, IgGC, and IgGD. Accordingly, the canine Fc fusion protein cIL-13Rα2-cIgGB-Fc is one species of the cIL-13Rα2-cFc genus, which also includes cIL-13Rα2-cIgGA-Fc, cIL-13Rα2-cIgGC-Fc, cIL-13Rα2-cIgGD-Fc and modified fusion proteins thereof.

As used herein a “canine Interleukin-13 receptor alpha 1-canine fragment crystallizable region fusion protein”, “canine Interleukin-13 receptor alpha 1-cFc fusion protein”, “canine IL-13Rα1-cFc fusion protein”, or “cIL-13Rα1-cFc fusion protein” are all used interchangeably and comprise the extracellular domain (ECD) of cIL-13Rα1 [or fragment of the ECD that binds canine Interleukin-13 (cIL-13)] connected to a canine IgG Fc (cFc) via a peptide linkage. In particular embodiments, a cIL-13Rα1-cFc fusion protein further comprises a canine hinge region that links the ECD of the cIL-13Rα1 (or fragment of the ECD that binds cIL-13) to the cFc. The cIL-13Rα1-cFc fusion protein can be generated from a chemically synthesized nucleic acid encoding the cIL-13Rα1 ECD (or fragment of the ECD that binds cIL-13) with the cFc (either with or without the linking hinge region) through genetic engineering.

As used herein a “canine Interleukin-13 receptor alpha 2-canine fragment crystallizable region fusion protein”, “canine Interleukin-13 receptor alpha 2-cFc fusion protein”, “canine IL-13Rα2-cFc fusion protein” or “cIL-13Rα2-cFc fusion protein” are all used interchangeably and comprise the extracellular domain (ECD) of cIL-13Rα2 [or fragment of the ECD that binds canine Interleukin-13 (cIL-13)] connected to a canine IgG Fc (cFc) via a peptide linkage. In particular embodiments, a cIL-13Rα2-cFc fusion protein further comprises a canine hinge region that links the ECD of the cIL-13Rα2 (or fragment of the ECD that binds cIL-13) to the cFc. The cIL-13Rα2-cFc fusion protein can be generated from a chemically synthesized nucleic acid encoding the cIL-13Rα2 ECD (or fragment of the ECD that binds cIL-13) with the cFc (either with or without the linking hinge region) through genetic engineering.

As used herein a cIL-13Rα2-cFc fusion protein comprising a “fragment of an ECD of cIL-13Rα2 that binds cIL-13” (or interchangeably, “a fragment thereof” of an ECD of the cIL-13Rα2 that binds cIL-13), has a binding affinity for cIL-13 that is at most a factor of 100 less than the binding affinity of the corresponding cIL-13Rα2-cFc fusion protein comprising the full length ECD, i.e., the dissociation constant is at most a factor of 10² higher. In certain embodiments, a cIL-13Rα2-cFc fusion protein comprising a fragment of an ECD of cIL-13Rα2 that binds cIL-13 has a binding affinity for cIL-13 that is at most a factor of 10 less than the binding affinity of the corresponding cIL-13Rα2-cFc fusion protein comprising the full length ECD, i.e., the dissociation constant is at most a factor of 10 higher. In still other embodiments, a cIL-13Rα2-cFc fusion protein comprising a fragment of an ECD of cIL-13Rα2 that binds cIL-13 has a binding affinity for cIL-13 that is at most a factor of 5 less than that of the binding affinity of the corresponding cIL-13Rα2-cFc fusion protein comprising the full length ECD, i.e., the dissociation constant is at most a factor of 5 higher.

As used herein a cIL-13Rα1-cFc fusion protein comprising a “fragment of an ECD of cIL-13Rα1 that binds cIL-13” (or interchangeably, “a fragment thereof” of the ECD of cIL-13Rα1 that binds cIL-13), has a binding affinity for cIL-13 that is at most a factor of 100 less than the binding affinity of the corresponding cIL-13Rα1-cFc fusion protein comprising the full length ECD, i.e., the dissociation constant is at most a factor of 10² higher. In certain embodiments, a cIL-13Rα1-cFc fusion protein comprising a fragment of an ECD of cIL-13Rα1 that binds cIL-13 has a binding affinity for cIL-13 that is at most a factor of 10 less than the binding affinity of the corresponding cIL-13Rα1-cFc fusion protein comprising the full length ECD, i.e., the dissociation constant is at most a factor of 10 higher. In still other embodiments, a cIL-13Rα1-cFc fusion protein comprising a fragment of an ECD of cIL-13Rα1 that binds cIL-13 has a binding affinity for cIL-13 that is at most a factor of 5 less than that of the binding affinity of the corresponding cIL-13Rα1-cFc fusion protein comprising the full length ECD, i.e., the dissociation constant is at most a factor of 5 higher.

As used herein a “homodimer” of a canine interleukin receptor-cFc fusion protein of the present invention is a dimer of two monomeric fusion proteins that minimally have the same ECD (or a fragment of that ECD that binds the corresponding ligand). The two monomeric fusion proteins generally also have the same cFc and the same hinge region. For example, when the canine Interleukin receptor-cFc fusion protein is a cIL-13Rα2-cFc fusion protein, the ECD is a cIL-13Rα2 ECD and the ligand is cIL-13. The two monomers of the homodimers are held together by disulfide bonds formed by the cysteine residues in the hinge region of each monomer. For example, a homodimer of a cIL-13Rα2-cFc fusion protein comprises two cIL-13Rα2-cFc fusion protein monomers and a homodimer of a cIL-13Rα1-cFc fusion protein comprises two cIL-13Rα1-cFc fusion protein monomers.

As used herein, the IgG Fc fusion proteins of the present invention, the antibodies, and/or the antigen binding fragments of the antibodies of the present invention “block” or are “blocking” or are “blocking the binding” of one binding partner to another binding partner (e.g., a receptor to its ligand). An antibody and/or fusion protein that blocks (partially or fully) the binding of two binding partners, e.g., the receptor to its ligand and vice versa, can be determined in standard binding assays (e.g., BIACore®, ELISA, or flow cytometry).

As used herein, the term “caninized antibody” refers to forms of IgG antibodies that contain amino acid sequences originating from both canine and non-canine (e.g., mouse, rat, or human) IgG antibodies, whereas, as used herein, the term “felinized antibody” refers to forms of IgG antibodies that contain amino acid sequences originating from both feline and non-feline (e.g., mouse, rat, or human) IgG antibodies. In general, the caninized antibody or felinized antibody will comprise substantially all of at least one or more typically, two variable domains in which all or substantially all of the hypervariable loops correspond to those of a non-canine or a non-feline immunoglobulin respectively (e.g., comprising 6 CDRs as exemplified below), and all or substantially all of the framework (FR) regions (and typically all or substantially all of the remaining frame) are those of a canine immunoglobulin or a feline immunoglobulin sequence, respectively. A modified canine frame comprises one or more amino acids changes as exemplified herein that further optimize the effectiveness of the caninized antibody, e.g., to increase its binding to its canine antigen and/or its ability to block the binding of that canine antigen to the canine antigen's natural binding partner. As exemplified below, a caninized antibody comprises both the three heavy chain CDRs and the three light chain CDRS from a mouse anti-canine antigen antibody together with a modified canine frame. As detailed below, the cFc of the caninized antibody can also comprise amino acid substitutions that lead to a greater half-life of the caninized antibody, i.e., have an increased binding affinity for its neonatal Fc receptor (FcRn) at pH 5.5-pH 6.5.

Similarly, a felinized antibody can comprise both the three heavy chain CDRs and the three light chain CDRS from e.g., a mouse, rat, or human, together with a feline frame or more commonly a modified feline frame. A modified feline frame comprises one or more amino acids changes that further optimize the effectiveness of the felinized antibody, e.g., to increase its binding to its feline antigen and/or its ability to block the binding of that feline antigen to the feline antigen's natural binding partner. Similarly, the fFc of the felinized antibody can also comprise amino acid substitutions that lead to a greater half-life of the felinized antibody i.e., have an increased binding affinity for its neonatal Fc receptor (FcRn) at pH 5.5-pH 6.5. As used herein a “fFc fusion protein” is used interchangeably with the term “feline IgG Fc fusion protein” and is an artificial protein that joins the fFc of a feline IgG antibody, which can include a hinge region, e.g., the IgG-1a hinge region-CH2-CH3, with another biologically active protein domain to generate a molecule with unique structure and therapeutic utility.

Caninized murine or rat anti-canine antibodies that bind canine interleukin-31 (cIL-31) or canine interleukin-31 receptor alpha (cIL-31RA or cIL-31Rα) include, but are not limited to antibodies for use in the present invention that comprise canine IgGA, IgGB, IgGC, or IgGD heavy chains, and modified forms of the canine IgGA, IgGB, IgGC, or IgGD heavy chains, including the modified Fcs that are disclosed herein, particularly those caninized antibodies that also show an increased binding affinity for its neonatal Fc receptor (FcRn) at pH 5.5-pH 6.5. As used herein, the term “caninized antibody to canine interleukin-31 receptor alpha” is used interchangeably with a “caninized cIL-31RA antibody” and a “cIL-31RA caninized antibody” and is a caninized antibody of a mammalian antibody raised in a non-canine mammal (e.g., a mouse or a rat) against cIL-31RA.

Typically, an antibody or antigen binding fragment of the antibodies of the invention retains at least 10% of its antigen binding activity (when compared to the parental antibody) when that activity is expressed on a molar basis. Preferably, an antibody or antigen binding fragment of the invention retains at least 20%, 50%, 70%, 80%, 90%, 95% or 100% or more of the antigen binding affinity as the parental antibody. It is also intended that an antibody or antigen binding fragment of the antibodies of the invention can include conservative or non-conservative amino acid substitutions (referred to as “conservative variants” or “function conserved variants” of the antibody) that do not substantially alter its biologic activity.

As used herein an “antipruritic agent” is a compound, macromolecule, and/or formulation that tends to inhibit, relieve, and/or prevent itching. Antipruritic agents are colloquially referred to as anti-itch drugs.

As used herein an “antipruritic antibody” is an antibody that can act as an antipruritic agent in an animal, including a mammal such as a human, a canine, and/or a feline, particularly with respect to atopic dermatitis. In particular embodiments, the antipruritic antibody binds to specific proteins in the IL-31 signaling pathway, such as IL-31 or its receptor IL-3 IRA. The binding of the antipruritic antibody to its corresponding antigen (e.g., IL-31 or IL-3 IRA) inhibits the binding of e.g., IL-31 with IL-3 IRA, and interferes with and/or prevents the successful signaling of this pathway, and thereby inhibits, relieves, and/or prevents the itching that is otherwise caused by the IL-31 signaling pathway.

As used herein an “anti-inflammatory agent” is a compound, macromolecule, and/or formulation that that reduces inflammation by blocking the interaction of certain substances in the body that cause inflammation. The anti-inflammatory agent can be a cFc fusion protein that can act as an anti-inflammatory agent in an animal, including a mammal such as a human, a canine, and/or a feline, particularly with respect to atopic dermatitis. In particular embodiments, the anti-inflammatory cFc fusion protein binds to specific proteins in the TL-4/TL-13 signaling pathway, such as TL-4 or TL-13. The binding of the anti-inflammatory cFc fusion protein to its corresponding antigen (e.g., IL-4) inhibits the binding of e.g., IL-4 with IL-4Rα, and interferes with and/or prevents the signaling of this pathway, thereby interfering with or preventing the chronic inflammation associated with atopic dermatitis. The combination of homodimers of the cIL-4Rα-cFc fusion protein with homodimers of the cIL-13Rα2-cFc fusion protein acts as an anti-inflammatory agent in the treatment of atopic dermatitis.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

Sequence identity refers to the degree to which the amino acids of two polypeptides are the same at equivalent positions when the two sequences are optimally aligned. As used herein one amino acid sequence is 100% “identical” to a second amino acid sequence when the amino acid residues of both sequences are identical. Accordingly, an amino acid sequence is 50% “identical” to a second amino acid sequence when 50% of the amino acid residues of the two amino acid sequences are identical. The sequence comparison is performed over a contiguous block of amino acid residues comprised by a given protein, e.g., a protein, or a portion of the polypeptide being compared. In particular embodiments, selected deletions or insertions that could otherwise alter the correspondence between the two amino acid sequences are taken into account.

Sequence similarity includes identical residues and nonidentical, biochemically related amino acids. Biochemically related amino acids that share similar properties and may be interchangeable.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes frequently can be made without altering the biological activity of the protein. Those of skill in this art recognize that, in general, single amino acid substitutions in non-essential regions of a polypeptide do not substantially alter biological activity [see, e.g., Watson et al., Molecular Biology of the Gene, The Benjamin/Cummings Pub. Co., p. 224 (4th Ed.; 1987)]. In addition, substitutions of structurally or functionally similar amino acids are less likely to disrupt biological activity. Exemplary conservative substitutions are set forth in Table A directly below.

TABLE A
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala; Gly |
| Ser (S) | Thr |
| Thr (T) | Ser |
| Trp (W) | Tyr; Phe |
| Tyr (Y) | Trp; Phe |
| Val (V) | Ile; Leu |

Function-conservative variants of the IgG Fc fusion proteins of the invention are also contemplated by the present invention. “Function-conservative variants,” as used herein, refers to the IgG Fc fusion proteins or antibodies in which one or more amino acid residues have been changed without altering a desired property, such an antigen affinity and/or specificity. Such variants include, but are not limited to, replacement of an amino acid with one having similar properties, such as the conservative amino acid substitutions of Table A above.

### Nucleic Acids

The present invention comprises the IgG Fc fusion proteins, antibodies, and antigen binding fragments of the antibodies of the present invention and compositions that comprise the IgG Fc fusion proteins, antibodies, and antigen binding fragments of the antibodies (see e.g., Examples below).

Also included in the present invention are the nucleic acids that encode the IgG Fc fusion proteins, the antibodies, and the antigen binding fragments of the antibodies provided of the present invention, comprising amino acid sequences that are at least about 70% identical, preferably at least about 80% identical, more preferably at least about 90% identical and most preferably at least about 95% identical (e.g., 95%, 96%, 97%, 98%, 99%, 100%) to the amino acid sequences of the caninized antibodies provided herein when the comparison is performed by a BLAST algorithm wherein the parameters of the algorithm are selected to give an exact match between the respective sequences over the entire length of the respective reference sequences, i.e., 95% identical means that 95% of the amino acids in the two sequences are identical. The present invention further provides nucleic acids that encode the fusion proteins and/or the immunoglobulin polypeptides which comprise nucleic acid sequences that are at least about 70% identical, preferably at least about 80% identical, more preferably at least about 90% identical and most preferably at least about 95% identical (e.g., 95%, 96%, 97%, 98%, 99%, 100%) to any of the reference nucleic acid sequences when the comparison is performed with a BLAST algorithm, wherein the parameters of the algorithm are selected to give the exact match, between the respective nucleotide sequences over the entire length of the respective reference sequences, i.e., at least 98% identical means that at least 98% of the nucleotides in the two nucleic acid sequences are identical, are also included in the present invention.

As used herein, nucleotide and amino acid sequence percent identity can be determined using C, MacVector (MacVector, Inc. Cary, NC 27519), Vector NTI (Informax, Inc. MD), Oxford Molecular Group PLC (1996) and the Clustal W algorithm with the alignment default parameters, and default parameters for identity. These commercially available programs can also be used to determine sequence similarity using the same or analogous default parameters. Alternatively, an Advanced Blast search under the default filter conditions can be used, e.g., using the GCG (Genetics Computer Group, Program Manual for the GCG Package, Version 7, Madison, Wisconsin) pileup program using the default parameters.

The following references relate to BLAST algorithms often used for sequence analysis: BLAST ALGORITHMS: Altschul, S. F., et al., J. Mol. Biol. 215:403-410 (1990); Gish, W., et al., Nature Genet. 3:266-272 (1993); Madden, T. L., et al., Meth. Enzymol. 266:131-141(1996); Altschul, S. F., et al., Nucleic Acids Res. 25:3389-3402 (1997); Zhang, J., et al., Genome Res. 7:649-656 (1997); Wootton, J. C., et al., Comput. Chem. 17:149-163 (1993); Hancock, J. M. et al., Comput. Appl. Biosci. 10:67-70 (1994); ALIGNMENT SCORING SYSTEMS: Dayhoff, M. O., et al., “A model of evolutionary change in proteins.” in Atlas of Protein Sequence and Structure, vol. 5, suppl. 3. M. O. Dayhoff (ed.), pp. 345-352, (1978); Natl. Biomed. Res. Found., Washington, DC; Schwartz, R. M., et al., “Matrices for detecting distant relationships.” in Atlas of Protein Sequence and Structure, vol. 5, suppl. 3.” (1978), M. O. Dayhoff (ed.), pp. 353-358 (1978), Natl. Biomed. Res. Found., Washington, DC; Altschul, S. F., J. Mol. Biol. 219:555-565 (1991); States, D. J., et al., Methods 3:66-70(1991); Henikoff, S., et al., Proc. Natl. Acad. Sci. USA 89:10915-10919 (1992); Altschul, S. F., et al., J. Mol. Evol. 36:290-300 (1993); ALIGNMENT STATISTICS: Karlin, S., et al., Proc. Natl. Acad. Sci. USA 87:2264-2268 (1990); Karlin, S., et al., Proc. Natl. Acad. Sci. USA 90:5873-5877 (1993); Dembo, A., et al., Ann. Prob. 22:2022-2039 (1994); and Altschul, S. F. “Evaluating the statistical significance of multiple distinct local alignments.” in Theoretical and Computational Methods in Genome Research (S. Suhai, ed.), pp. 1-14, Plenum, New York (1997).

The IgG Fc fusion proteins, the antibodies, and the antigen binding fragments of the antibodies of the present invention can be produced recombinantly by methods that are known in the field. Mammalian cell lines available as hosts for expression of the antibodies or fragments disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the heavy chain or antigen-binding portion or fragment thereof, the light chain and/or antigen-binding fragment thereof are introduced into mammalian host cells, the antibodies are produced by culturing the host cells for a period of time sufficient to allow for expression of the antibody in the host cells or, more preferably, secretion of the antibody into the culture medium in which the host cells are grown.

Antibodies can be recovered from the culture medium using standard protein purification methods. Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

### Pharmaceutical Compositions and Administration

To prepare pharmaceutical or sterile compositions comprising the IgG Fc fusion proteins, the antibodies, and the antigen binding fragments of the antibodies of the present invention, can be admixed with a pharmaceutically acceptable carrier or excipient. [See, e.g., Remington's Pharmaceutical Sciences and U.S. Pharmacopeia: National Formulary, Mack Publishing Company, Easton, PA (1984)].

Formulations of therapeutic and diagnostic agents may be prepared by mixing with acceptable carriers, excipients, or stabilizers in the form of, e.g., lyophilized powders, slurries, aqueous solutions or suspensions [see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY]. In one embodiment, pharmaceutical compositions comprising the IgG Fc fusion proteins, the antibodies, and/or the antigen binding fragments of the antibodies of the present invention are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the IgG Fc fusion proteins, the antibodies, and the antigen binding fragments of the antibodies of the compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, the IgG Fc fusion proteins, the antibodies, or the antigen binding fragments of the antibodies of the present invention exhibiting high therapeutic indices are desirable. The data obtained from these cell culture assays and animal studies can be used in formulating a range of dosage for use in the subjects, e.g., canines or felines. The dosage of such compounds lies preferably within a range of circulating concentrations that include the ED₅₀ with little or no toxicity. The dosage may vary within this range depending upon the dosage form employed and the route of administration.

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. In particular embodiments, pharmaceutical compositions comprising the IgG Fc fusion proteins, the antibodies, and/or the antigen binding fragments of the antibodies of the present invention can be administered by an invasive route such as by injection. In further embodiments of the invention, pharmaceutical compositions comprising the IgG Fe fusion proteins, the antibodies, and/or the antigen binding fragments of the antibodies of the present invention are administered intravenously, subcutaneously, intramuscularly, intraarterially, or by inhalation, aerosol delivery. Administration by non-invasive routes (e.g., orally; for example, in a pill, capsule or tablet) is also within the scope of the present invention.

Compositions can be administered with medical devices known in the art. For example, a pharmaceutical composition of the invention can be administered by injection with a hypodermic needle, including, e.g., a prefilled syringe or autoinjector. The pharmaceutical compositions disclosed herein may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The pharmaceutical compositions disclosed herein may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternatively, one may administer compositions comprising the IgG Fc fusion proteins, the antibodies, and/or the antigen binding fragments of the antibodies of the present invention in a local rather than systemic manner, often in a depot or sustained release formulation.

The administration regimen depends on several factors, including the serum or tissue turnover rate of the therapeutic IgG Fc fusion proteins, the antibodies, and/or the antigen binding fragments of the antibodies, the level of symptoms, the immunogenicity of the therapeutic IgG Fc fusion proteins, the antibodies, and/or the antigen binding fragments of the antibodies and the accessibility of the target cells in the biological matrix. Preferably, the administration regimen delivers sufficient therapeutic IgG Fc fusion proteins, the antibodies, and/or the antigen binding fragments of the antibodies to effect improvement in the target disease/condition state, while simultaneously minimizing undesired side effects. Accordingly, the amount of biologic delivered depends in part on the particular therapeutic antibodies, and/or fusion proteins and the severity of the condition being treated. Guidance in selecting appropriate doses of therapeutic antibodies is available [see, e.g., Wawrzynczak Antibody Therapy, Bios Scientific Pub. Ltd, Oxfordshire, U K (1996); Kresina (ed.) Monoclonal Antibodies, Cytokines and Arthritis, Marcel Dekker, New York, NY (1991); Bach (ed.) Monoclonal Antibodies and Peptide Therapy in Autoimmune Diseases, Marcel Dekker, New York, NY (1993); Baert, et al. New Engl. J. Med. 348:601-608 (2003); Milgrom et al. New Engl. J. Med. 341:1966-1973 (1999); Slamon et al. New Engl. J. Med. 344:783-792 (2001); Beniaminovitz et al. New Engl. J. Med. 342:613-619 (2000); Ghosh et al. New Engl. J. Med. 348:24-32 (2003); Lipsky et al. New Engl. J Med. 343:1594-1602 (2000)].

Determination of the appropriate dose is made by the veterinarian, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of the symptoms.

The compositions comprising the IgG Fc fusion proteins, the antibodies, and/or the antigen binding fragments of the antibodies of the present invention, either alone or with the antibodies used in the present invention may be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more [see, e.g., Yang, et al. New Engl. J Med. 349:427-434 (2003); Herold, et al. New Engl. J. Med. 346:1692-1698 (2002); Liu, et al. J. Neurol. Neurosurg. Psych. 67:451-456 (1999); Portielji, et al. Cancer Immunol. Immunother. 52:133-144 (2003)]. Doses may also be provided to achieve a pre-determined target concentration of the IgG Fc fusion proteins, the antibodies, and/or the antigen binding fragments of the antibodies of the present invention in the subject's serum, such as 0.1, 0.3, 1, 3, 10, 30, 100, 300 μg/ml or more. In other embodiments, the IgG Fc fusion proteins, the antibodies, and/or the antigen binding fragments of the antibodies of the present invention are administered subcutaneously or intravenously, on a weekly, biweekly, “every 4 weeks,” monthly, bimonthly, or quarterly basis at 10, 20, 50, 80, 100, 200, 500, 1000 or 2500 mg/subject.

As used herein, “inhibit” or “treat” or “treatment” includes a postponement of development of the symptoms associated with a disorder or condition and/or a reduction in the severity of the symptoms of such disorder or condition. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject (e.g., a canine) with a disorder, condition and/or symptom, or with the potential to develop such a disorder, disease or symptom.

As used herein, the terms “therapeutically effective amount”, “therapeutically effective dose” and “effective amount” refer to an amount of the IgG Fc fusion proteins, the antibodies, and/or the antigen binding fragments of the antibodies of the present invention that, when administered alone or in combination with an additional therapeutic agent to a cell, tissue, or subject, e.g., canine or feline, is effective to cause a measurable improvement in one or more symptoms of a disease or condition or the progression of such disease or condition. A therapeutically effective dose further refers to that amount of the IgG Fc fusion proteins, the antibodies, and/or the antigen binding fragments of the antibodies sufficient to result in at least partial amelioration of symptoms, e.g., treatment, healing, prevention or amelioration of the relevant medical condition, or an increase in rate of treatment, healing, prevention or amelioration of such conditions. When applied to a combination, a therapeutically effective dose refers to combined amounts of the active ingredients that result in the therapeutic effect, whether administered in combination, serially, or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess severity of the condition.

### EXAMPLES

### Example 1

### Canine FcRn Receptor Cloning and Expression

The subunits of canine FcRn proteins [IgG Receptor and Transporter (FCGRT) and β2-microglobulin (B2M)] have been described [Bergeron, et al., Vet Immunol and Immunopathol. 157:31-41 (2014)]. Their sequences can be found in the National Center for Biotechnology Information databases and are provided below. The DNA encoding the FCGRT, which was engineered with a c-terminal [GGGGS; SEQ ID NO: 46]×3 linker, AviTag, and 8×His tag, and B2M, which was prepared without a c-terminal tag, were cloned into pcDNA3.4 vectors with the artificial signaling peptide MGWSCIILFLVATATGVHS [SEQ ID NO: 47]. Equal mass amounts of each vector was co-transfected into Expi293F cells. Two-step purification was performed by HisTrapFF™ Crude and HiLoad™ 26/600 Superdex™ 200 prep grade columns to purify the FcRn heterodimer.

XP_533618.2 IgG receptor FcRn large subunit p51
isoform X2 [Canis lupus familiaris]:
[SEQ ID NO: 48]
MGVPRPRSWGLGFLLFLLPTLRADSHLSLLYHLTAVSAPPPGTPAFWAS
GWLGPQQYLSYNNLRAQAEPYGAWVWENQVSWYWEKETTDLRTKEGLFL
EALKALGDGGPYTLQGLLGCELGPDNTSVPVAKFALNGEDFMTFDPKLG
TWNGDWPETETVSKRWMQQAGAVSKERTFLLYSCPQRLLGHLERGRGNL
EWKEPPSMRLKARPGSPGFSVLTCSAFSFYPPELQLRFLRNGLAAGSGE
GDFGPNGDGSFHAWSSLTVKSGDEHHYRCLVQHAGLPQPLTVELESPAK
SSVPVVGIVIGFLLLTAVAVGGALLWRRMRKGLPAPWMSLRGDDVGALL
PTPGVPKDADS
XP535458.1 PREDICTED: similar to beta-2-
microglobulin (B2M) precursor isoform 2
[Canis familiars]
[SEQ ID NO: 49]
MAPRPALATAGFLALLLILLAACRLDAVQHPPKIQVYSRHPAENGKPNF
LNCYVSGFHPPEIEIDLLKNGKEMKAEQTDLSFSKDWTFYLLVHTEFTP
NEQDEFSCRVKHVTLSEPQIVKWDRDN

### Example 2

### In Vitro Binding to FcRn Receptors

The binding of antibodies and IgG Fc fusion proteins to FcRn proteins was determined by Octet™ HTX using Streptavidin (SA) biosensors. First, biotin-labeled FcRn/32M was loaded onto pre-hydrated SA biosensors for 60 s at a concentration of 10 μg/mL. Second, the biosensors were placed into pH 6.0 TBS/Casein buffer for the blocking phase for 180 s. Third, for the association phase, FcRn/B2M loaded biosensors were placed into 2-fold serial dilutions from 500 nM down to 7.8 nM of antibody or IgG Fc fusion protein in pH 6.0 or 7.4 TBS/Casein buffer for 15 s. Finally, the biosensors were placed into either pH 6.0 or pH 7.4 TBS/Casein buffer for the dissociation phase for 60 s. Analysis was performed using Octet Data Analysis 12.0 software and curves were fitted using a 1:1 binding model.

### Example 3

### Canine and Feline IgG Fc Regions

Provided below are the amino acid sequences of the four canine Fc regions and the three feline Fc regions, as well as the corresponding amino acid sequences of the modified Fc regions that comprise two amino acid residue substitutions, D31A and N63A, as exemplified in the amino acid sequence of SEQ ID NO: 3 of IgG-B compared to the amino acid sequence of SEQ ID NO: 4 of IgG-Bm. These two amino acid residue substitutions serve to significantly diminish the antibody-dependent cytotoxicity (ADCC) and complement-dependent cytotoxicity (CDC) of the naturally occurring canine IgG-B [see, U.S. Pat. No. 10,106,607 B2, the contents of which are hereby incorporated by reference in their entirety]. Following the EU numbering scheme of Sequences of Proteins of Immunological Interest, 5th ed., Kabat et al., National Institutes of Health, Bethesda, Md. (1991), D31A and N63A correspond to amino acid positions D265A and N297A, respectively.

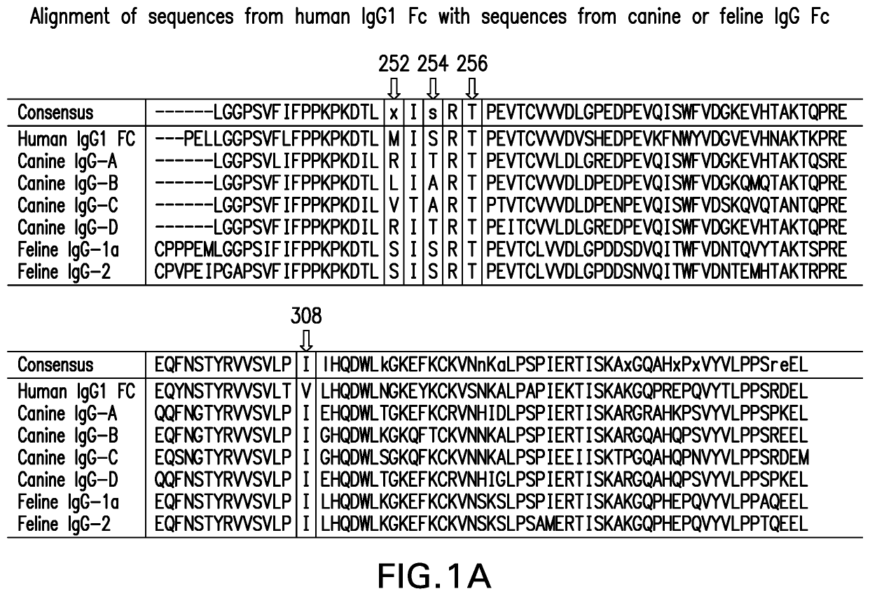

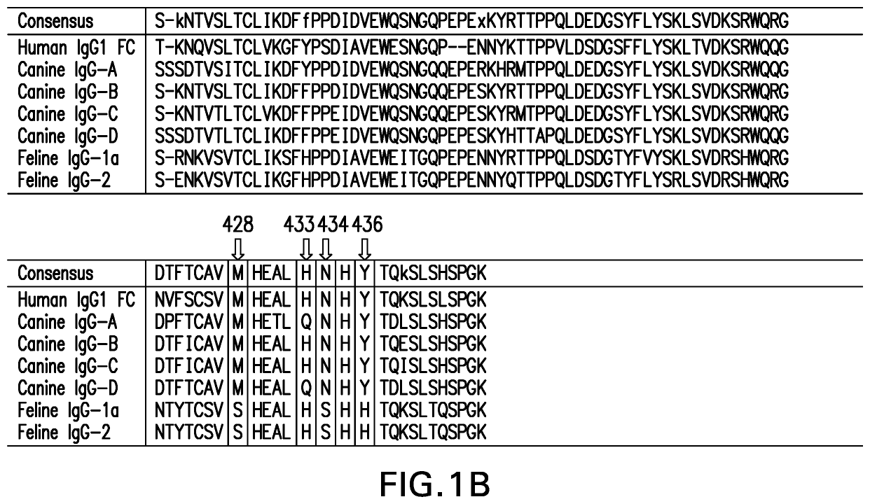

The alignment of the amino acid sequences of the Fc regions from canine IgG-A [SEQ ID NO: 1], canine IgG-B [SEQ ID NO: 3], canine IgG-C [SEQ ID NO: 5], canine IgG-D [SEQ ID NO: 7], feline IgG-1a [SEQ ID NO: 52], feline IgG-2 [SEQ ID NO: 53], human IgG1 Fc [SEQ ID NO: 54], and the consensus sequence of these seven amino acid sequences [SEQ ID NO: 55] are depicted in FIG. 1. The amino acid sequences of the Fc regions of feline IgG-1a and feline IgG-2 Fc regions further includes two additional N-terminal amino acid residues coming from their respective hinge regions, as denoted by Strietzel et al., [Vet Immunol & Immunopathol., 158:214-223 (2014)]. The amino acid sequences for the feline Fc regions are: [SEQ ID NO: 9] for feline IgG-1a, [SEQ ID NO: 50] for feline IgG-1b, and [SEQ ID NO: 11] for feline IgG-2, according to Strietzel et al., supra.

Canine IgG Fc
IgG-A
[SEQ ID NO: 1]
LGGPSVLIFPPKPKDILRITRTPEVTCVVLDLGREDPEVQISWFVDGKEVHTAKTQSREQQFNGT
YRVVSVLPIEHQDWLTGKEFKCRVNHIDLPSPIERTISKARGRAHKPSVYVLPPSPKELSSSDTV
SITCLIKDFYPPDIDVEWQSNGQQEPERKHRMTPPQLDEDGSYFLYSKLSVDKSRWQQGDPFTCA
VMHETLQNHYTDLSLSHSPGK
IgG-Am
[SEQ ID NO: 2]
LGGPSVLIFPPKPKDILRITRTPEVTCVVLALGREDPEVQISWFVDGKEVHTAKTQSREQQFAGT
YRVVSVLPIEHQDWLTGKEFKCRVNHIDLPSPIERTISKARGRAHKPSVYVLPPSPKELSSSDTV
SITCLIKDFYPPDIDVEWQSNGQQEPERKHRMTPPQLDEDGSYFLYSKLSVDKSRWQQGDPFTCA
VMHETLQNHYTDLSLSHSPGK
IgG-B
[SEQ ID NO: 3]
LGGPSVFIFPPKPKDTLLIARTPEVTCVVVDLDPEDPEVQISWFVDGKQMQTAKTQPREEQFNGT
YRVVSVLPIGHQDWLKGKQFTCKVNNKALPSPIERTISKARGQAHQPSVYVLPPSREELSKNTVS
LTCLIKDFFPPDIDVEWQSNGQQEPESKYRTTPPQLDEDGSYFLYSKLSVDKSRWQRGDTFICAV
MHEALHNHYTQESLSHSPGK
IgG-Bm
[SEQ ID NO: 4]
LGGPSVFIFPPKPKDTLLIARTPEVTCVVVALDPEDPEVQISWFVDGKQMQTAKTQPREEQFAGT
YRVVSVLPIGHQDWLKGKQFTCKVNNKALPSPIERTISKARGQAHQPSVYVLPPSREELSKNTVS
LTCLIKDFFPPDIDVEWQSNGQQEPESKYRTTPPQLDEDGSYFLYSKLSVDKSRWQRGDTFICAV
MHEALHNHYTQESLSHSPGK
IgG-C
[SEQ ID NO: 5]
LGGPSVFIFPPKPKDILVTARTPTVTCVVVDLDPENPEVQISWFVDSKQVQTANTQPREEQSNGT
YRVVSVLPIGHQDWLSGKQFKCKVNNKALPSPIEEIISKTPGQAHQPNVYVLPPSRDEMSKNTVT
LTCLVKDFFPPEIDVEWQSNGQQEPESKYRMTPPQLDEDGSYFLYSKLSVDKSRWQRGDTFICAV
MHEALHNHYTQISLSHSPGK
IgG-Cm
[SEQ ID NO: 6]
LGGPSVFIFPPKPKDILVTARTPTVTCVVVALDPENPEVQISWFVDSKQVQTANTQPREEQSAGT
YRVVSVLPIGHQDWLSGKQFKCKVNNKALPSPIEEIISKTPGQAHQPNVYVLPPSRDEMSKNTVT
LTCLVKDFFPPEIDVEWQSNGQQEPESKYRMTPPQLDEDGSYFLYSKLSVDKSRWQRGDTFICAV
MHEALHNHYTQISLSHSPGK
IgG-D
[SEQ ID NO: 7]
LGGPSVFIFPPKPKDILRITRTPEITCVVLDLGREDPEVQISWFVDGKEVHTAKTQPREQQFNST
YRVVSVLPIEHQDWLTGKEFKCRVNHIGLPSPIERTISKARGQAHQPSVYVLPPSPKELSSSDTV
TLTCLIKDFFPPEIDVEWQSNGQPEPESKYHTTAPQLDEDGSYFLYSKLSVDKSRWQQGDTFTCA
VMHEALQNHYTDLSLSHSPGK
IgG-Dm
[SEQ ID NO: 8]
LGGPSVFIFPPKPKDILRITRTPEITCVVLALGREDPEVQISWFVDGKEVHTAKTQPREQQFAST
YRVVSVLPIEHQDWLTGKEFKCRVNHIGLPSPIERTISKARGQAHQPSVYVLPPSPKELSSSDTV
TLTCLIKDFFPPEIDVEWQSNGQPEPESKYHTTAPQLDEDGSYFLYSKLSVDKSRWQQGDTFTCA
VMHEALQNHYTDLSLSHSPGK
Feline IgG Fc
IgG-1a
[SEQ ID NO: 9]
PPEMLGGPSIFIFPPKPKDTLSISRTPEVTCLVVDLGPDDSDVQITWFVDNTQVYTAKTSPREEQ
FNSTYRVVSVLPILHQDWLKGKEFKCKVNSKSLPSPIERTISKAKGQPHEPQVYVLPPAQEELSR
NKVSVTCLIKSFHPPDIAVEWEITGQPEPENNYRTTPPQLDSDGTYFVYSKLSVDRSHWQRGNTY
TCSVSHEALHSHHTQKSLTQSPGK
IgG-1am
[SEQ ID NO: 10]
PPEMLGGPSIFIFPPKPKDTLSISRTPEVTCLVVALGPDDSDVQITWFVDNTQVYTAKTSPREEQ
FASTYRVVSVLPILHQDWLKGKEFKCKVNSKSLPSPIERTISKAKGQPHEPQVYVLPPAQEELSR
NKVSVTCLIKSFHPPDIAVEWEITGQPEPENNYRTTPPQLDSDGTYFVYSKLSVDRSHWQRGNTY
TCSVSHEALHSHHTQKSLTQSPGK
IgG-1b
[SEQ ID NO: 50]
PPEMLGGPSIFIFPPKPKDTLSISRTPEVTCLVVDLGPDDSDVQITWFVDNTQVYTAKTSPREEQ
FNSTYRVVSVLPILHQDWLKGKEFKCKVNSKSLPSPIERTISKDKGQPHEPQVYVLPPAQEELSR
NKVSVTCLIEGFYPSDIAVEWEITGQPEPENNYRTTPPQLDSDGTYFLYSRLSVDRSRWQRGNTY
TCSVSHEALHSHHTQKSLTQSPGK
IgG-1bm
[SEQ ID NO: 51]
PPEMLGGPSIFIFPPKPKDTLSISRTPEVTCLVVALGPDDSDVQITWFVDNTQVYTAKTSPREEQ
FASTYRVVSVLPILHQDWLKGKEFKCKVNSKSLPSPIERTISKDKGQPHEPQVYVLPPAQEELSR
NKVSVTCLIEGFYPSDIAVEWEITGQPEPENNYRTTPPQLDSDGTYFLYSRLSVDRSRWQRGNTY
TCSVSHEALHSHHTQKSLTQSPGK
IgG-2
[SEQ ID NO: 11]
VPEIPGAPSVFIFPPKPKDTLSISRTPEVTCLVVDLGPDDSNVQITWFVDNTEMHTAKTRPREEQ
FNSTYRVVSVLPILHQDWLKGKEFKCKVNSKSLPSAMERTISKAKGQPHEPQVYVLPPTQEELSE
NKVSVTCLIKGFHPPDIAVEWEITGQPEPENNYQTTPPQLDSDGTYFLYSRLSVDRSHWQRGNTY
TCSVSHEALHSHHTQKSLTQSPGK
IgG-2m
[SEQ ID NO: 12]
VPEIPGAPSVFIFPPKPKDTLSISRTPEVTCLVVALGPDDSNVQITWFVDNTEMHTAKTRPREEQ
FASTYRVVSVLPILHQDWLKGKEFKCKVNSKSLPSAMERTISKAKGQPHEPQVYVLPPTQEELSE
NKVSVTCLIKGFHPPDIAVEWEITGQPEPENNYQTTPPQLDSDGTYFLYSRLSVDRSHWQRGNTY
TCSVSHEALHSHHTQKSLTQSPGK
cIgG-A hinge region
[SEQ ID NO: 13]
FNECRCTDTPPCPVPEP
cIgG-B hinge region
[SEQ ID NO: 14]
PKRENGRVPRPPDCPKCPT¹PEM
cIgG-C hinge region
[SEQ ID NO: 15]
AKECECKCNCNNCPCPGCGL
cIgG-D hinge region
[SEQ ID NO: 16]
PKESTCKCISPCPVPES
IgG-1a Fc plus 2 amino acids residues
[SEQ ID NO: 52]
CPPPEMLGGPSIFIFPPKPKDTLSISRTPEVTCLVVDLGPDDSDVQITWFVDNTQVYTAKTSPRE
EQFNSTYRVVSVLPILHQDWLKGKEFKCKVNSKSLPSPIERTISKAKGQPHEPQVYVLPPAQEEL
SRNKVSVTCLIKSFHPPDIAVEWEITGQPEPENNYRTTPPQLDSDGTYFVYSKLSVDRSHWQRGN
TYTCSVSHEALHSHHTQKSLTQSPGK
IgG-2 Fc plus 2 amino acids residues
[SEQ ID NO: 53]
CPVPEIPGAPSVFIFPPKPKDTLSISRTPEVTCLVVDLGPDDSNVQITWFVDNTEMHTAKTRPRE
EQFNSTYRVVSVLPILHQDWLKGKEFKCKVNSKSLPSAMERTISKAKGQPHEPQVYVLPPTQEEL
SENKVSVTCLIKGFHPPDIAVEWEITGQPEPENNYQTTPPQLDSDGTYFLYSRLSVDRSHWQRGN
TYTCSVSHEALHSHHTQKSLTQSPGK
Human IgG1 Fc
[SEQ ID NO: 54]
PELLGGPSVFLFPPKPKDTLMISRTPEVTCVVVDVSHEDPEVKENWYVDGVEVHNAKTKPREEQY
NSTYRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVESCS
VMHEALHNHYTQKSLSLSPGK
Consensus sequence:
[SEQ ID NO: 55]
LGGPSVFIFPPKPKDTLXISRTPEVTCVVVDLGPEDPEVQISWFVDGKEVHTAKTQPREEQFNST
YRVVSVLPILHQDWLKGKEFKCKVNNKALPSPIERTISKAXGQAHXPXVYVLPPSREELSKNXTV
SLTCLIKDFFPPDIDVEWQSNGQPEPEXKYRTTPPQLDEDGSYFLYSKLSVDKSRWQRGDTFTCA
VMHEALHNHYTQKSLSHSPGK

FIG. 1 depicts the EU numbering scheme according to Sequences of Proteins of Immunological Interest, 5th ed., Kabat et al., supra, which is used herein. Amino acid residue numbers 252, 254, 256, 308, 428, 433, 434, and 436 are amino acid residue positions comprised by the amino acid sequences of the cFc regions and fFc regions of the present invention that are specifically identified in FIG. 1 and accordingly, follow the EU numbering scheme of Kabat et al., supra. ¹ This threonine (T) also has been identified as an alanine (A).

### Example 4

### Construction, Expression, and Purification of Modified Canine Igg Fc Fusion Proteins and Antibodies

### BACKGROUND

Atopic dermatitis (AD) is a relapsing pruritic and chronic inflammatory skin disease in humans and companion animals that is characterized by immune system dysregulation and epidermal barrier abnormalities. Both interleukin-4 (IL-4) and interleukin-13 (IL-13) are part of a signaling pathway involved in the chronic inflammatory skin disease in atopic dermatitis. IL-4 binds to a heterodimeric receptor, which comprises a monomer of the common γc chain (γc) and a monomer of the IL-4 receptor alpha (IL-4Rα). IL-13 binds to a heterodimeric receptor comprising a monomer of the IL-13 receptor alpha 1(IL-13Rα1) and a monomer of the IL-4Rα. Blocking the binding of IL-4 to IL-4Rα and IL-13 to IL-13Rα1 blocks the concomitant skin inflammation. An IgG Fc fusion protein comprising either the extracellular domain (ECD) of either IL-13Rα1 (e.g., the cIL-13Rα1-cFc fusion protein) or IL-13Rα2 (e.g., the cIL-13Rα2-cFc fusion protein) can bind IL-13. An IgG Fc fusion protein comprising the ECD of IL-4Rα (e.g., the canine IL-4Rα-cFc fusion protein) can bind IL-4Rα. Combining the both the IgG Fc fusion protein comprising the ECD of either IL-13Rα1 or IL-13R Rα2 with the IgG Fc fusion protein comprising the ECD of IL-4Rα can block the signaling through IL-4Rα and thereby, ameliorate the chronic inflammatory skin disease of atopic dermatitis.

The binding of interleukin-31 (IL-31) to its receptor, the IL-31 receptor a (IL-31RA), initiates the pruritic effect of atopic dermatitis in both humans and dogs. Blocking the binding of IL-31 to IL-3 IRA with an antibody that binds IL-31RA serves to block the signaling through IL-3 IRA and ameliorates the pruritic effect of atopic dermatitis. Extending the half-life of the IgG Fc IL-13Rα1 or IL-13R Rα2 IgG fusion proteins and/or IgG Fc IL-4Rα, and/or extending the half-life of IL-3 IRA antibodies allows the corresponding potential drugs to be used at relatively lower doses and/or with reduced dosing frequency.

### Methods

Amino acid residue modifications were genetically engineered in the canine Fc regions of the heavy chain of IgG antibodies and cFc-fusion proteins to improve their affinity for the neonatal Fc receptor FcRn at pH 6. The nucleic acid encoding the artificial signaling peptide MGWSCIILFLVATATGVHS [SEQ ID NO: 16] and the amino acid sequence of the heavy chain if the antibody comprised the hinge region of canine IgG-B Fc and the canine IgG-Bm Fc region. Alternatively, the extracellular-domain of canine IL-13Rα2 was fused to the canine IgG-B Fc hinge region and the canine IgG-B Fc region. Nucleic acids encoding the antibodies (canine heavy chain as described above or the corresponding canine light chain) or cFc-fusion proteins were cloned into pcDNA3.4 vectors. Each vector was transfected into ExpiCHO cells and the harvested supernatant was purified on recombinant Protein A affinity columns. The proteins were eluted in 0.1 M Glycine-HCl, pH 2.7 and then adjusted to pH 6.0 using 1 M Tris Buffer, pH 8.0.

CANINEIL-13Rα1 AND IL-13Rα2
Canine IL-13Rα1
[SEQ ID NO: 17]
VLPAKPENISCIFYYEENFTCTWSPEKEASYTWYKVKRTYSYGYKSDICS
TDNSTRGNHASCSFLPPTITNPDNYTIQVEAQNADGIMKSDITYWNLDAI
MKIEPPEIFSVKSVLGIKRMLQIKWIRPVLAPHSSTLKYTLRFRTINSAY
WMEVNFTKEDIDRDETYNLTELQAFTEYVMTLRCAPAESMFWSGWSQEKV
GTTEEEAPYGLDLWRVLKPAMVDGRRPVQLMWKKATGAPVLEKALGYNIW
YFPENNTNLTETVNTTNQTHELYLGGKTYWVYVVSYNSLGESPVATLRIP
ALNEKTFQCIEAMQACLTQDQLVVEWQSSAPEVDTWMVEWFPDVDSEPSS
FSWESVSQARNWTIQKDELKPLWCYNISVYPVLRDRVGQPYSTQAYVQEG
IPSAGPVTQADSIGVKTVTITWKEIPKSKRNGFIKNYTIFYQAEDGKEFS
KTVNSNILQYRLESLTRRTSYSLQVMASTNAGGINGTKINFKTLSIS
Canine IL-13Rα2
[SEQ ID NO: 18]
SMLSNAEIKVNPPQDFEIVDPGYLGYLSLQWQPPLFPDNFKECTIEYELK
YRNIDSENWKTIITKNLHYKDGFDLNKGIEAKINTLLPAQCTNGSEVRSS
WAETTYWTSPQGNRETKIQDMDCVYYNWQYLVCSWKPGMGVHFDTNYQLF
YWYEGLDHSAECTDYIKVNGKNMGCRFPYLESSDYKDFYICVNGSSESQP
IRPSYFIFQLQNIVKPMPPDYLSLTVKNSEEINLKWNMPKGPIPAKCFIY
EIEFTEDGTTWVTTTVENEIQITRTSNESQKLCFLVRSKVNIYCSDDGIW
SEWSDEQCWKGDIWKET

### Amino Acid Sequences of Exemplary Modified Canine IL-13Rα1 and IL-13Rα2 IgG Fc Fusion Proteins

Accordingly, in order to increase the half-life of the cFc-fusion protein, the canine IgG-B Fc regions of the fusion proteins of the present invention were modified relative to the corresponding wild type canine IgG-B Fc. Such a modified cFc region comprises one or more amino acid substitutions relative to wild type cFc region at one or more amino acid residues 252, 254, 256, 433, 434, and 436 [numbered according to Sequences of Proteins of Immunological Interest, 5th ed., Kabat et al., National Institutes of Health, Bethesda, Md. (1991); the EU Index]. Particular examples include modifications at one or more of the following positions: L252Y, A254T, T256D, T256E, I308P, H433K, H433L, N434H, N434F, N434Y, and Y436T, numbered as shown in FIG. 1, and provided in Table 1 below.

cIL-13Rα2-cIgGB-Fc-Y N434Y
[SEQ ID NO: 19]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCTNGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDFFPPDIDVEWQSNGQQEPESKYRTT
PPQLDEDGSYFLYSKLSVDKSRWQRGDTFICAVMHEALHYHYTQESLSHSPGK
cIL-13Rα2-cIgGB-Fc-F N434F
[SEQ ID NO: 20]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCTNGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDFFPPDIDVEWQSNGQQEPESKYRTT
PPQLDEDGSYFLYSKLSVDKSRWQRGDTFICAVMHEALHFHYTQESLSHSPGK
cIL-13Rα2-cIgGB-Fc-L H433L
[SEQ ID NO: 21]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCTNGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDEFPPDIDVEWQSNGQQEPESKYRTT
PPQLDEDGSYFLYSKLSVDKSRWQRGDTFICAVMHEALLNHYTQESLSHSPGK
cIL-13Rα2-cIgGB-Fc-K H433K
[SEQ ID NO: 22]
MGWSCIILFLVATATGVHSSMLSNAEIKVNPPQDFEIVDPGYLGYLSLQWQPPLFPDNEKECTIE
YELKYRNIDSENWKTIITKNLHYKDGFDLNKGIEAKINTLLPAQCINGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDFFPPDIDVEWQSNGQQEPESKYRTT
PPQLDEDGSYFLYSKLSVDKSRWQRGDTFICAVMHEALKNHYTQESLSHSPGK
cIL-13Rα2-cIgGB-Fc-T Y436T
[SEQ ID NO: 23]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCINGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDFFPPDIDVEWQSNGQQEPESKYRTT
PPQLDEDGSYFLYSKLSVDKSRWQRGDTFICAVMHEALHNHTTQESLSHSPGK
cIL-13Rα2-cIgGB-Fc-LY H433L/N434Y
[SEQ ID NO: 24]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCTNGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDEFPPDIDVEWQSNGQQEPESKYRTT
PPQLDEDGSYFLYSKLSVDKSRWQRGDTFICAVMHEALLYHYTQESLSHSPGK
cIL-13Rα2-cIgGB-Fc-KY H433K/N434Y
[SEQ ID NO: 25]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCTNGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDFFPPDIDVEWQSNGQQEPESKYRTT
PPQLDEDGSYFLYSKLSVDKSRWQRGDTFICAVMHEALKYHYTQESLSHSPGK
cIL-13Rα2-cIgGB-Fc-KF H433K/N434F
[SEQ ID NO: 26]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCTNGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDFFPPDIDVEWQSNGQQEPESKYRTT
PPQLDEDGSYFLYSKLSVDKSRWQRGDTFICAVMHEALKFHYTQESLSHSPGK
cIL-13Rα2-cIgGB-Fc-LF H433L/N434F
[SEQ ID NO: 27]
MGWSCIILFLVATATGVHSSMLSNAEIKVNPPQDFEIVDPGYLGYLSLQWQPPLFPDNEKECTIE
YELKYRNIDSENWKTIITKNLHYKDGFDLNKGIEAKINTLLPAQCTNGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDEFPPDIDVEWQSNGQQEPESKYRTT
PPQLDEDGSYFLYSKLSVDKSRWQRGDTFICAVMHEALLFHYTQESLSHSPGK
cIL-13Rα2-cIgGB-Fc-KYT H433K/N434Y/Y436T
[SEQ ID NO: 28]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCTNGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDFFPPDIDVEWQSNGQQEPESKYRTT
PPQLDEDGSYFLYSKLSVDKSRWQRGDTFICAVMHEALKYHTTQESLSHSPGK
cIL-13Rα2-cIgGB-Fc-LYT H433L/N434Y/Y436T
[SEQ ID NO: 29]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCTNGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDFFPPDIDVEWQSNGQQEPESKYRTT
PPQLDEDGSYFLYSKLSVDKSRWQRGDTFICAVMHEALLYHTTQESLSHSPGK
cIL-13Rα2-cIgGB-Fc-LFT H433L/N434F/Y436T
[SEQ ID NO: 30]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCTNGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDFFPPDIDVEWQSNGQQEPESKYRTT
PPQLDEDGSYFLYSKLSVDKSRWQRGDTFICAVMHEALLFHTTQESLSHSPGK
cIL-13Rα2-cIgGB-Fc-KFT H433K/N434F/Y436T
[SEQ ID NO: 31]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCINGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDFFPPDIDVEWQSNGQQEPESKYRTT
PPQLDEDGSYFLYSKLSVDKSRWQRGDTFICAVMHEALKFHTTQESLSHSPGK
cIL-13Rα2-cIgGB-Fc-PY I308P/N434Y
[SEQ ID NO: 32]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCTNGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPPGHQDWLKGKQFTCKVNNKALPSP
IERTISKARGQAHQPSVYVLPPSREELSKNTVSLTCLIKDFFPPDIDVEWQSNGQQEPESKYRTT
PPQLDEDGSYFLYSKLSVDKSRWQRGDTFICAVMHEALHYHYTQESLSHSPGK
cIL-13Rα2-cIgGB-Fc-DPY T256D/I308P/N434Y
[SEQ ID NO: 33]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCTNGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARDPEVTCVVVDL
DPEDPEVQISWFVDGKQMQTAKTQPREEQFNGTYRVVSVLPPGHQDWLKGKQFTCKVNNKALPSP
IERTISKARGQAHQPSVYVLPPSREELSKNTVSLTCLIKDFFPPDIDVEWQSNGQQEPESKYRTT
PPQLDEDGSYFLYSKLSVDKSRWQRGDTFICAVMHEALHYHYTQESLSHSPGK
cIL-13Rα2-cIgGB-Fc-DY T256D/N434Y
[SEQ ID NO: 34]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCTNGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARDPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDEFPPDIDVEWQSNGQQEPESKYRTT
PPQLDEDGSYFLYSKLSVDKSRWQRGDTFICAVMHEALHYHYTQESLSHSPGK
cIL-13Rα2-cIgGB-Fc-YT N434Y/Y436T
[SEQ ID NO: 35]
MGWSCIILFLVATATGVHSSMLSNAEIKVNPPQDFEIVDPGYLGYLSLQWQPPLFPDNFKECTIE
YELKYRNIDSENWKTIITKNLHYKDGFDLNKGIEAKINTLLPAQCTNGSEVRSSWAETTYWTSPQ
GNRETKIQDMDCVYYNWQYLVCSWKPGMGVHFDTNYQLFYWYEGLDHSAECTDYIKVNGKNMGCR
FPYLESSDYKDFYICVNGSSESQPIRPSYFIFQLQNIVKPMPPDYLSLTVKNSEEINLKWNMPKG
PIPAKCFIYEIEFTEDGTTWVTTTVENEIQITRTSNESQKLCFLVRSKVNIYCSDDGIWSEWSDE
QCWKGDIWKETPKRENGRVPRPPDCPKCPAPEMLGGPSVFIFPPKPKDTLLIARTPEVTCVVVDL
DPEDPEVQISWFVDGKQMQTAKTQPREEQFNGTYRVVSVLPIGHQDWLKGKQFTCKVNNKALPSP
IERTISKARGQAHQPSVYVLPPSREELSKNTVSLTCLIKDFFPPDIDVEWQSNGQQEPESKYRTT
PPQLDEDGSYFLYSKLSVDKSRWQRGDTFICAVMHEALHYHTTQESLSHSPGK

TABLE 1
BINDING AFFINITY AT pH 6.0 AND 7.4 OF cIL-13Rα2-cIgGB-Fc
FUSION PROTEINS COMPRISING THE AMINO ACID
RESIDUE SUBSTITUTIONS INDICATED

| | Amino acid | pH 6.0 | pH 7.4 |
|---|---|---|---|
| Sample ID | substitution | KD (M) | KD (M) |
| cIL-13Ra2-cIgGB-Fc-Y | N434Y | 1.10E−10 | 1.87E−07 |
| cIL-13Ra2-cIgGB-Fc-KYT | H433K/N434Y/ | 2.58E−09 | 2.53E−07 |
| | Y436T | | |
| cIL-13Ra2-cIgGB-Fc-F | N434F | 9.30E−10 | 2.29E−07 |
| cIL-13Ra2-cIgGB-Fc-LYT | H433L/N434Y/ | 3.32E−09 | 1.27E−07 |
| | Y436T | | |
| cIL-13Ra2-cIgGB-Fc-L | H433L | 1.23E−08 | 7.21E−06 |
| cIL-13Ra2-cIgGB-Fc-LFT | H433L/N434F/ | 2.87E−09 | 9.05E−07 |
| | Y436T | | |
| cIL-13Ra2-cIgGB-Fc-K | H433K | 7.55E−09 | 6.26E−07 |
| cIL-13Ra2-cIgGB-Fc-KFT | H433K/N434F/ | 9.31E−07 | 1.36E−07 |
| | Y436T | | |
| cIL-13Ra2-cIgGB-Fc-T | Y436T | 5.95E−09 | 6.12E−06 |
| cIL-13Ra2-cIgGB-Fc-PY | I308P/N434Y | 3.00E−10 | 2.04E−08 |
| cIL-13Ra2-cIgGB-Fc-LY | H433L/N434Y | 2.35E−09 | 2.98E−07 |
| cIL-13Ra2-cIgGB-Fc-DPY | T256D/I308P/ | 3.40E−10 | 1.98E−08 |
| | N434Y | | |
| cIL-13Ra2-cIgGB-Fc-KY | H433K/N434Y | 4.60E−13 | 2.96E−07 |
| cIL-13Ra2-cIgGB-Fc-DY | T256D/N434Y | 8.70E−10 | 9.18E−08 |
| cIL-13Ra2-cIgGB-Fc-KF | H433K/N434F | 9.70E−10 | 4.14E−07 |
| cIL-13Ra2-cIgGB-Fc-YT | N434Y/Y436T | 1.39E−09 | 2.33E−07 |
| cIL-13Ra2-cIgGB-Fc-LF | H433L/N434F | 2.27E−09 | 2.26E−07 |
| cIL-13Ra2-Fc | None | 1.69E−08 | 7.02E−06 |
| cIL-13Ra2-Fc-YTE | L252Y/A254T/ | 6.59E−09 | 3.63E−07 |
| | T256E | | |

### Amino Acid Sequences of Exemplary Antibody Half-Life Enhancing Modifications

In order to increase the half-life of caninized mouse anti-canine IL-3 IRA antibodies, the canine IgG-Bm Fc regions of the canine heavy chains of the present invention were modified relative to the corresponding canine IgG-Bm Fc. Such a modified cFc region comprises one or more amino acid substitutions relative to wild type cFc region at one or more amino acid residues 252, 256, 433, and 434. [numbered according to Sequences of Proteins of Immunological Interest, 5th ed., Kabat et al., National Institutes of Health, Bethesda, Md. (1991); the EU Index]. Particular examples include modifications at one or more of the following positions: L252Y, T256D, T256E, H433K, H433L, N434F, and N434Y, as numbered as shown in FIG. 1, and provided in Table 2 below.

c218D9VH4-cIgGBm-F N434F
[SEQ ID NO: 36]
MGWSCIILFLVATATGVHSEVTLQESGPGLVKPSQTLSLTCSFSGFSLSTFGRGVGWIRQRPGRG
LEWMGHIWWDDDKYYNPALKSRLSITKDTAKNQVFLQLSSMTTEDTAVYYCARIAGGLRRAPYAM
DSWGQGTLVTVSSASTTAPSVFPLAPSCGSTSGSTVALACLVSGYFPEPVTVSWNSGSLTSGVHT
FPSVLQSSGLYSLSSMVTVPSSRWPSETFTCNVAHPASKTKVDKPVPKRENGRVPRPPDCPKCPA
PEMLGGPSVFIFPPKPKDTLLIARTPEVTCVVVALDPEDPEVQISWFVDGKQMQTAKTQPREEQF
AGTYRVVSVLPIGHQDWLKGKQFTCKVNNKALPSPIERTISKARGQAHQPSVYVLPPSREELSKN
TVSLTCLIKDFFPPDIDVEWQSNGQQEPESKYRTTPPQLDEDGSYFLYSKLSVDKSRWQRGDTFI
CAVMHEALHFHYTQESLSHSPGK
c218D9VH4-cIgGBm-Y N434Y
[SEQ ID NO: 37]
MGWSCIILFLVATATGVHSEVTLQESGPGLVKPSQTLSLTCSFSGFSLSTFGRGVGWIRQRPGRG
LEWMGHIWWDDDKYYNPALKSRLSITKDTAKNQVFLQLSSMTTEDTAVYYCARIAGGLRRAPYAM
DSWGQGTLVTVSSASTTAPSVFPLAPSCGSTSGSTVALACLVSGYFPEPVTVSWNSGSLTSGVHT
FPSVLQSSGLYSLSSMVTVPSSRWPSETFTCNVAHPASKTKVDKPVPKRENGRVPRPPDCPKCPA
PEMLGGPSVFIFPPKPKDTLLIARTPEVTCVVVALDPEDPEVQISWFVDGKQMQTAKTQPREEQF
AGTYRVVSVLPIGHQDWLKGKQFTCKVNNKALPSPIERTISKARGQAHQPSVYVLPPSREELSKN
TVSLTCLIKDFFPPDIDVEWQSNGQQEPESKYRTTPPQLDEDGSYFLYSKLSVDKSRWQRGDTFI
CAVMHEALHYHYTQESLSHSPGK
c218D9VH4-cIgGBm-KF H433K/N434F
[SEQ ID NO: 38]
MGWSCIILFLVATATGVHSEVTLQESGPGLVKPSQTLSLTCSFSGFSLSTFGRGVGWIRQRPGRG
LEWMGHIWWDDDKYYNPALKSRLSITKDTAKNQVFLQLSSMTTEDTAVYYCARIAGGLRRAPYAM
DSWGQGTLVTVSSASTTAPSVEPLAPSCGSTSGSTVALACLVSGYFPEPVTVSWNSGSLTSGVHT
FPSVLQSSGLYSLSSMVTVPSSRWPSETFTCNVAHPASKTKVDKPVPKRENGRVPRPPDCPKCPA
PEMLGGPSVFIFPPKPKDTLLIARTPEVTCVVVALDPEDPEVQISWFVDGKQMQTAKTQPREEQF
AGTYRVVSVLPIGHQDWLKGKQFTCKVNNKALPSPIERTISKARGQAHQPSVYVLPPSREELSKN
TVSLTCLIKDFFPPDIDVEWQSNGQQEPESKYRTTPPQLDEDGSYFLYSKLSVDKSRWQRGDTFI
CAVMHEALKFHYTQESLSHSPGK
c218D9VH4-cIgGBm-KY H433K/N434Y
[SEQ ID NO: 39]
MGWSCIILFLVATATGVHSEVTLQESGPGLVKPSQTLSLTCSFSGFSLSTFGRGVGWIRQRPGRG
LEWMGHIWWDDDKYYNPALKSRLSITKDTAKNQVFLQLSSMTTEDTAVYYCARIAGGLRRAPYAM
DSWGQGTLVTVSSASTTAPSVEPLAPSCGSTSGSTVALACLVSGYFPEPVTVSWNSGSLTSGVHT
FPSVLQSSGLYSLSSMVTVPSSRWPSETFTCNVAHPASKTKVDKPVPKRENGRVPRPPDCPKCPA
PEMLGGPSVFIFPPKPKDTLLIARTPEVTCVVVALDPEDPEVQISWFVDGKQMQTAKTQPREEQF
AGTYRVVSVLPIGHQDWLKGKQFTCKVNNKALPSPIERTISKARGQAHQPSVYVLPPSREELSKN
TVSLTCLIKDFFPPDIDVEWQSNGQQEPESKYRTTPPQLDEDGSYFLYSKLSVDKSRWQRGDTFI
CAVMHEALKYHYTQESLSHSPGK
c218D9VH4-cIgGBm-LF H433L/N434F
[SEQ ID NO: 40]
MGWSCIILFLVATATGVHSEVTLQESGPGLVKPSQTLSLTCSFSGFSLSTFGRGVGWIRQRPGRG
LEWMGHIWWDDDKYYNPALKSRLSITKDTAKNQVFLQLSSMTTEDTAVYYCARIAGGLRRAPYAM
DSWGQGTLVTVSSASTTAPSVFPLAPSCGSTSGSTVALACLVSGYFPEPVTVSWNSGSLTSGVHT
FPSVLQSSGLYSLSSMVTVPSSRWPSETFTCNVAHPASKTKVDKPVPKRENGRVPRPPDCPKCPA
PEMLGGPSVFIFPPKPKDTLLIARTPEVTCVVVALDPEDPEVQISWFVDGKQMQTAKTQPREEQF
AGTYRVVSVLPIGHQDWLKGKQFTCKVNNKALPSPIERTISKARGQAHQPSVYVLPPSREELSKN
TVSLTCLIKDFFPPDIDVEWQSNGQQEPESKYRTTPPQLDEDGSYFLYSKLSVDKSRWQRGDTFI
CAVMHEALLFHYTQESLSHSPGK
c218D9VH4-cIgGBm-LY H433L/N434Y
[SEQ ID NO: 41]
MGWSCIILFLVATATGVHSEVTLQESGPGLVKPSQTLSLTCSFSGFSLSTFGRGVGWIRQRPGRG
LEWMGHIWWDDDKYYNPALKSRLSITKDTAKNQVFLQLSSMTTEDTAVYYCARIAGGLRRAPYAM
DSWGQGTLVTVSSASTTAPSVEPLAPSCGSTSGSTVALACLVSGYFPEPVTVSWNSGSLTSGVHT
FPSVLQSSGLYSLSSMVTVPSSRWPSETFTCNVAHPASKTKVDKPVPKRENGRVPRPPDCPKCPA
PEMLGGPSVFIFPPKPKDTLLIARTPEVTCVVVALDPEDPEVQISWFVDGKQMQTAKTQPREEQF
AGTYRVVSVLPIGHQDWLKGKQFTCKVNNKALPSPIERTISKARGQAHQPSVYVLPPSREELSKN
TVSLTCLIKDFFPPDIDVEWQSNGQQEPESKYRTTPPQLDEDGSYFLYSKLSVDKSRWQRGDTFI
CAVMHEALLYHYTQESLSHSPGK
c218D9VH4-cIgGBm-YD L252Y/T256D
[SEQ ID NO: 42]
MGWSCIILFLVATATGVHSEVTLQESGPGLVKPSQTLSLTCSFSGFSLSTFGRGVGWIRQRPGRG
LEWMGHIWWDDDKYYNPALKSRLSITKDTAKNQVFLQLSSMTTEDTAVYYCARIAGGLRRAPYAM
DSWGQGTLVTVSSASTTAPSVEPLAPSCGSTSGSTVALACLVSGYFPEPVTVSWNSGSLTSGVHT
FPSVLQSSGLYSLSSMVTVPSSRWPSETFTCNVAHPASKTKVDKPVPKRENGRVPRPPDCPKCPA
PEMLGGPSVFIFPPKPKDTLYIARDPEVTCVVVALDPEDPEVQISWFVDGKQMQTAKTQPREEQF
AGTYRVVSVLPIGHQDWLKGKQFTCKVNNKALPSPIERTISKARGQAHQPSVYVLPPSREELSKN
TVSLTCLIKDFFPPDIDVEWQSNGQQEPESKYRTTPPQLDEDGSYFLYSKLSVDKSRWQRGDTFI
CAVMHEALHNHYTQESLSHSPGK
c218D9VH4-cIgGBm-H N434H
[SEQ ID NO: 43]
MGWSCIILFLVATATGVHSEVTLQESGPGLVKPSQTLSLTCSFSGFSLSTFGRGVGWIRQRPGRG
LEWMGHIWWDDDKYYNPALKSRLSITKDTAKNQVFLQLSSMTTEDTAVYYCARIAGGLRRAPYAM
DSWGQGTLVTVSSASTTAPSVEPLAPSCGSTSGSTVALACLVSGYFPEPVTVSWNSGSLTSGVHT
FPSVLQSSGLYSLSSMVTVPSSRWPSETFTCNVAHPASKTKVDKPVPKRENGRVPRPPDCPKCPA
PEMLGGPSVFIFPPKPKDTLLIARTPEVTCVVVALDPEDPEVQISWFVDGKQMQTAKTQPREEQF
AGTYRVVSVLPIGHQDWLKGKQFTCKVNNKALPSPIERTISKARGQAHQPSVYVLPPSREELSKN
TVSLTCLIKDFFPPDIDVEWQSNGQQEPESKYRTTPPQLDEDGSYFLYSKLSVDKSRWQRGDTFI
CAVMHEALHHHYTQESLSHSPGK
c218D9VH4
[SEQ ID NO: 44]
EVTLQESGPGLVKPSQTLSLTCSFSGFSLSTFGRGVGWIRQRPGRGLEWMGHIWWDDDKYYNPAL
KSRLSITKDTAKNQVFLQLSSMTTEDTAVYYCARIAGGLRRAPYAMDSWGQGTLVTVSS
c218D9VL3-cCK
[SEQ ID NO: 45]
DIVMTQTPLSLSVSPGETASISCRASENIYSSLAWFRQKPGQSPQLLVYAATNLADGVPDRESGS
GSGTDYTLRISRVEADDTGVYYCQHFRDTPPTFGQGTKLEIKRNDAQPAVYLFQPSPDQLHTGSA
SVVCLLNSFYPKDINVKWKVDGVIQDTGIQESVTEQDKDSTYSLSSTLTMSSTEYLSHELYSCEI
THKSLPSTLIKSFQRSECQRVD

TABLE 2
BINDING AFFINITY AT pH 6.0 AND 7.4 OF CANINIZED
ANTIBODIES TO CANINE IL-31RA COMPRISING THE
AMINO ACID RESIDUE SUBSTITUTIONS INDICATED

| | Amino Acid | pH 6.0 | pH 7.4 |
|---|---|---|---|
| Sample ID | substitution | KD (nM) | KD (nM) |
| c218D9-H4L3-cIgGBm | None | 1.11E−09 | No Detectable |
| | | | Binding |
| c218D9-H4L3-cIgGBm-F | N434F | 1.80E−10 | 9.02E−09 |
| c218D9-H4L3-cIgGBm-Y | N434Y | 6.70E−11 | 5.42E−09 |
| c218D9-H4L3-cIgGBm-KF | H433K/N434F | 1.00E−10 | 3.19E−09 |
| c218D9-H4L3-cIgGBm-KY | H433K/N434Y | 1.50E−11 | 3.40E−09 |
| c218D9-H4L3-cIgGBm-LF | H433L/N434F | 6.60E−11 | 1.58E−08 |
| c218D9-H4L3-cIgGBm-LY | H433L/N434Y | 1.70E−10 | 1.12E−08 |
| c218D9-H4L3-cIgGBm-YD | L252Y/T256D | 7.70E−12 | 1.05E−07 |
| c218D9-H4L3-cIgGBm-H | N434H | 3.40E−10 | 1.42E−08 |

SEQUENCE LISTING TABLE

| SEQ | | SEQ | |
|---|---|---|---|
| ID | DESCRIPTION | ID | DESCRIPTION |
| 1 | IgG-A Fc | 2 | IgG-Am Fc |
| 3 | IgG-B Fc | 4 | IgG-Bm Fc |
| 5 | IgG-C Fc | 6 | IgG-Cm Fc |
| 7 | IgG-D Fc | 8 | IgG-Dm Fc |
| 9 | IgG-la Fc | 10 | IgG-lam Fc |
| 11 | IgG-2 Fc | 12 | IgG-2m Fc |
| 13 | cIgG-A hinge region | 14 | cIgG-B hinge region |
| 15 | cIgG-C hinge region | 16 | cIgG-D hinge region |
| 17 | canine-13Rα1-cIgG | 18 | canine-13Rα2-cIgG |
| | fusion protein | | fusion protein |
| 19 | cIL-13Rα2-cIgGB-Fc-Y | 20 | cIL-13Rα2-cIgGB-Fc-F |
| | (N434Y) | | (N434F) |
| 21 | cIL-13Rα2-cIgGB-Fc-L | 22 | cIL-13Rα2-cIgGB-Fc-K |
| | (H433L) | | (H433K) |
| 23 | cIL-13Rα2-cIgGB-Fc-T | 24 | cIL-13Rα2-cIgGB-Fc-LY |
| | (Y436T) | | (H433L/N434Y) |
| 25 | cIL-13Rα2-cIgGB-Fc-KY | 26 | cIL-13Rα2-cIgGB-Fc-KF |
| | (H433K/N434Y) | | (H433K/N434F) |
| 27 | cIL-13Rα2-cIgGB-Fc-LF | 28 | cIL-13Rα2-cIgGB-Fc-KYT |
| | (H433L/N434F) | | (H433K/N434Y/Y436T) |
| 29 | cIL-13Rα2-cIgGB-Fc-LYT | 30 | cIL-13Rα2-cIgGB-Fc-LFT |
| | (H433L/N434Y/Y436T) | | (H433L/N434F/Y436T) |
| 31 | cIL-13Rα2-cIgGB-Fc-KFT | 32 | cIL-13Rα2-cIgGB-Fc-PY |
| | (H433K/N434F/Y436T) | | (I308P/N434Y) |
| 33 | cIL-13Rα2-cIgGB-Fc-DPY | 34 | cIL-13Rα2-cIgGB-Fc-DY |
| | (T256D/I308P/N434Y) | | (T256D/N434Y) |
| 35 | cIL-13Rα2-cIgGB-Fc-YT | 36 | c218D9VH4-cIgGBm-F |
| | (N434Y/Y436T) | | (N434F) |
| 37 | c218D9VH4-cIgGBm-Y | 38 | c218D9VH4-cIgGBm-KF |
| | (N434Y) | | (H433K/N434F) |
| 39 | c218D9VH4-cIgGBm-KY | 40 | c218D9VH4-cIgGBm-LF |
| | (H433K/N434Y) | | (H433L/N434F) |
| 41 | c218D9VH4-cIgGBm-LY | 42 | c218D9VH4-cIgGBm-YD |
| | (H433L/N434Y) | | (L252Y/T256D) |
| 43 | c218D9VH4-cIgGBm-H | 44 | c218D9VH4 |
| | (N434H) | | |
| 45 | c218D9VL3-cCK | | |
| 46 | GGGGS | 47 | MGWSCIILFLVATATGVHS |
| 48 | IgG receptor FcRn large | 49 | beta-2-microglobulin (B2M) |
| | subunit p51 | | precursor isoform 2 |
| 50 | IgG-lb | 51 | IgG-1bm Fc |
| 52 | IgG-1a Fc plus 2 amino | 53 | IgG-2 Fc plus 2 amino |
| | acids residues | | acids residues |
| 54 | Human IgG1 Fc | 55 | Consensus IgG Fc |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. An antibody or an IgG Fc fusion protein comprising a fragment crystallizable region (Fc region) that comprises one or more amino acid substitutions, wherein said one or more amino acid residue substitutions is at an amino acid residue position selected from the group consisting of 252, 254, 256, 308, 433, 434, 436, or any combination thereof, wherein the amino acid residue positions are numbered according to the EU index as in Kabat:
wherein when the substitution is at amino acid residue position 252, the substitution is with a tyrosine residue;
wherein when the substitution is at amino acid residue position 254, the substitution is with a threonine residue;
wherein when the substitution is at amino acid residue position 256, the substitution is with an aspartic acid residue or a glutamic acid residue;
wherein when the substitution is at amino acid residue position 308, the substitution is with a proline residue;
wherein when the substitution is at amino acid residue position 433, the substitution is with a lysine residue or a leucine residue;
wherein when the substitution is at amino acid residue position 434, the substitution is with a phenylalanine residue, a histidine residue, or a tyrosine residue;
wherein when the substitution is at amino acid residue position 436, the substitution is with a threonine residue;
wherein the antibody comprises a heavy chain and a light chain; and
wherein the Fc region is selected from the group consisting of a canine Fc region (cFc) and a feline Fc region (fFc).

2. The antibody or the IgG Fc fusion protein of claim 1, wherein the cFc or the fFc comprises two or more amino acid residue substitutions at the amino acid residue positions selected from the group consisting of:
the substitution at amino acid residue position 252, with a tyrosine residue and the substitution at amino acid residue position 256, with an aspartic acid residue;
the substitution at amino acid residue position 256, with an aspartic acid residue and the substitution at amino acid residue position 434, with a tyrosine residue;
the substitution at amino acid residue position 308, with a proline residue and the substitution at amino acid residue position 434, with a tyrosine residue;
the substitution at amino acid residue position 433, with a lysine residue and the substitution at amino acid residue position 434, with a phenylalanine residue;
the substitution at amino acid residue position 433, with a lysine residue and the substitution at amino acid residue position 434, with a tyrosine residue;
the substitution at amino acid residue position 433, with a leucine residue and the substitution at amino acid residue position 434, with a phenylalanine residue;
the substitution at amino acid residue position 433, with a leucine residue and the substitution at amino acid residue position 434, with a tyrosine residue;
the substitution at amino acid residue position 434, with a tyrosine residue and the substitution at amino acid residue position 436, with a threonine residue;
the substitution at amino acid residue position 252, with a tyrosine residue, the substitution at amino acid residue position 254, with a threonine residue, and the substitution at amino acid residue position 256, with a glutamic acid residue;
the substitution at amino acid residue position 256, with an aspartic acid residue, the substitution at amino acid residue position 308, with a proline residue, and the substitution at amino acid residue position 434, with a tyrosine residue;
the substitution at amino acid residue position 433, with a lysine residue, the substitution at amino acid residue position 434, with a phenylalanine residue, and the substitution at amino acid residue position 436, with a threonine residue;
the substitution at amino acid residue position 433, with a lysine residue, the substitution at amino acid residue position 434, with a tyrosine residue, and the substitution at amino acid residue position 436, with a threonine residue;
the substitution at amino acid residue position 433, with a leucine residue, the substitution at amino acid residue position 434, with a phenylalanine residue, and the substitution at amino acid residue position 436, with a threonine residue; and
the substitution at amino acid residue position 433, with a leucine residue, the substitution at amino acid residue position 434, with a tyrosine residue, and the substitution at amino acid residue position 436 with a threonine residue.

3. The antibody or the IgG Fc fusion protein of claim 1, wherein the fFc is selected from the group consisting of an IgG-1a Fc, an IgG-1am Fc, IgG-1b Fc, an IgG-1bm Fc, an IgG-2 Fc, and an IgG-2m Fc.

4. The antibody or the IgG Fc fusion protein of claim 3, wherein the fFc comprises an amino acid sequence selected from the group consisting of SEQ ID NO: 9, an amino acid sequence that has at least 90%, 95%, 97%, 98%, 99% identity to SEQ ID NO: 9, SEQ ID NO: 10, an amino acid sequence that has at least 90%, 95%, 97%, 98%, 99% identity to SEQ ID NO: 10, SEQ ID NO: 11, an amino acid sequence that has at least 90%, 95%, 97%, 98%, 99% identity to SEQ ID NO: 11, SEQ ID NO: 12, an amino acid sequence that has at least 90%, 95%, 97%, 98%, 99% identity to SEQ ID NO: 12, SEQ ID NO: 50, an amino acid sequence that has at least 90%, 95%, 97%, 98%, 99% identity to SEQ ID NO: 50, SEQ ID NO: 51, and an amino acid sequence that has at least 90%, 95%, 97%, 98%, 99% identity to SEQ ID NO: 51; and wherein the fFc retains said one or more amino acid substitutions.

5. The antibody or the IgG Fc fusion protein of claim 1, wherein the cFc is selected from the group consisting of an IgG-A Fc, an IgG-Am Fc, an IgG-B Fc, an IgG-Bm Fc, an IgG-C Fc, an IgG-Cm Fc, an IgG-D Fc, and an IgG-Dm Fc.

6. The antibody or the IgG Fc fusion protein of claim 5, wherein the cFc comprises an amino acid sequence selected from the group consisting of SEQ ID NO: 1, an amino acid sequence that has at least 90%, 95%, 97%, 98%, 99% identity to SEQ ID NO: 1, SEQ ID NO: 2, an amino acid sequence that has at least 90%, 95%, 97%, 98%, 99% identity to SEQ ID NO: 2; SEQ ID NO: 3, an amino acid sequence that has at least 90%, 95%, 97%, 98%, 99% identity to SEQ ID NO: 3, SEQ ID NO: 4, an amino acid sequence that has at least 90%, 95%, 97%, 98%, 99% identity to SEQ ID NO: 4, SEQ ID NO: 5, an amino acid sequence that has at least 90%, 95%, 97%, 98%, 99% identity to SEQ ID NO: 5, SEQ ID NO: 6, an amino acid sequence that has at least 90%, 95%, 97%, 98%, 99% identity to SEQ ID NO: 6, SEQ ID NO: 7, an amino acid sequence that has at least 90%, 95%, 97%, 98%, 99% identity to SEQ ID NO: 7, SEQ ID NO: 8, and an amino acid sequence that has at least 90%, 95%, 97%, 98%, 99% identity to SEQ ID NO: 8; and wherein the cFc retains said one or more amino acid substitutions.

7. The antibody or the IgG Fc fusion protein of claim 1, wherein the cFc further comprises a canine hinge region that comprises an amino acid sequence that has at least 80%, 85%, 90%, or 95% identity with the amino acid sequence selected from the group consisting of SEQ ID NO: 13, SEQ ID NO: 14, SEQ ID NO: 15, and SEQ ID NO: 16.

8. The antibody or the IgG Fc fusion protein of claim 7, wherein the cFc further comprises a canine hinge region that comprises the amino acid sequence selected from the group consisting of SEQ ID NO: 13, SEQ ID NO: 14, SEQ ID NO: 15, and SEQ ID NO: 16.

9. The antibody or the IgG Fc fusion protein of claim 1, that has an increased half-life compared to the half-life of the corresponding antibody or the IgG Fc fusion protein that comprises the corresponding wild type canine Fc or feline Fc.

10. The antibody or the IgG Fc fusion protein of claim 1, that has an enhanced binding affinity for their neonatal Fc receptor (FcRn) at moderately acidic pH.

11. The IgG Fc fusion protein of claim 1, which is a canine IL-13Rα1-canine IgG fusion protein, that further comprises an amino acid sequence of SEQ ID NO: 17, or an amino acid sequence that has at least 90%, 95%, 97%, 98%, 99% identity to SEQ ID NO: 17; and wherein the cFc retains said one or more amino acid substitutions.

12. The IgG Fc fusion protein of claim 1, which is a canine-13Rα2-canine IgG fusion protein, that further comprises an amino acid sequence of SEQ ID NO: 18, or an amino acid sequence that has at least 90%, 95%, 97%, 98%, 99% identity to SEQ ID NO: 18; and wherein the cFc retains said one or more amino acid substitutions.

13. The canine-13Rα2-canine IgG fusion protein of claim 12, that comprises an amino acid sequence selected from the group consisting of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35, or an amino acid sequence selected from the group consisting of an amino acid sequence that comprises at least 90%, 95%, 97%, 98%, 99% identity to SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35.

14. A nucleic acid that encodes the antibody or the IgG fusion protein of claim 1.

15. An expression vector that comprises the nucleic acid of claim 14.

16. A host cell that comprises the expression vector of claim 15.

17. The antibody of claim 1, wherein the antibody is selected from the group consisting of a canine antibody or a caninized antibody.

18. The caninized antibody of claim 17, that is a caninized antibody to canine interleukin-31 receptor alpha (cIL-31RA); and wherein the heavy chain comprises a variable region comprising the amino acid sequence of SEQ ID NO: 44.

19. The caninized antibody to cIL-31RA of claim 18, wherein the heavy chain comprises an amino acid sequence selected from the group consisting of SEQ ID NO: 36, SEQ ID NO: 37, SEQ ID NO: 38, SEQ ID NO: 39, SEQ ID NO: 40, SEQ ID NO: 41, SEQ ID NO: 42, and SEQ ID NO: 43, or an amino acid sequence that comprises at least 90%, 95%, 97%, 98%, 99% identity to SEQ ID NO: 36, SEQ ID NO: 37, SEQ ID NO: 38, SEQ ID NO: 39, SEQ ID NO: 40, SEQ ID NO: 41, SEQ ID NO: 42, and SEQ ID NO: 43.

20. The caninized antibody to cIL-31 RA of claim 18; wherein the light chain of the caninized antibody comprises the amino acid sequence of SEQ ID NO: 45 or comprises at least 90%, 95%, 97%, 98%, 99% identity to SEQ ID NO: 45.

21. A nucleic acid that encodes the light chain of the caninized antibody of claim 20.

22. A nucleic acid that encodes the heavy chain of the caninized antibody of claim 17.

23. An expression vector that comprises the nucleic acid of claim 21.

24. A host cell that comprises the expression vector of claim 23.

25. A pharmaceutical composition comprising the canine-13Rα1-canine IgG fusion protein of claim 11 and a pharmaceutically acceptable carrier.

26. A method of treating a canine that has atopic dermatitis comprising administering to the canine the pharmaceutical composition of claim 25.

27. A nucleic acid that encodes the canine-13Rα1-canine IgG fusion protein of claim 11.

28. A nucleic acid that encodes the canine-13Rα2-canine IgG fusion protein of claim 12.


SEQUENCE LISTING (55 sequences)

SEQ ID NO: 1 - protein, 216 aa, Canis lupus
  source 1..216: note = Canis familiaris
LGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 2 - protein, 216 aa, synthetic construct
  REGION 1..216: note = Modified canine
LGGPSVLIFPPKPKDILRITRTPEVTCVVLALGREDPEVQISWFVDGKEVHTAKTQSREQQFAGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 3 - protein, 215 aa, Canis lupus
  source 1..215: note = Canis familiaris
LGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 4 - protein, 215 aa, synthetic construct
  REGION 1..215: note = Modified Canine
LGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 5 - protein, 215 aa, Canis lupus; identical to SEQ ID NO: 4
  source 1..215: note = Canis familiaris

SEQ ID NO: 6 - protein, 215 aa, synthetic construct
  REGION 1..215: note = Modified canine
LGGPSVFIFPPKPKDILVTARTPTVTCVVVALDPENPEVQISWFVDSKQVQTANTQPREEQSAGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 7 - protein, 216 aa, Canis lupus
  source 1..216: note = Canis familiaris
LGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 8 - protein, 216 aa, synthetic construct
  REGION 1..216: note = Modified canine
LGGPSVFIFPPKPKDILRITRTPEITCVVLALGREDPEVQISWFVDGKEVHTAKTQPREQQFASTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 9 - protein, 219 aa, Felis catus
PPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 10 - protein, 219 aa, synthetic construct
  REGION 1..219: note = Modified feline
PPEMLGGPSIFIFPPKPKDTLSISRTPEVTCLVVALGPDDSDVQITWFVDNTQVYTAKTSPREEQFASTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 11 - protein, 219 aa, Felis catus
VPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 12 - protein, 219 aa, synthetic construct
  REGION 1..219: note = Modified Feline
VPEIPGAPSVFIFPPKPKDTLSISRTPEVTCLVVALGPDDSNVQITWFVDNTEMHTAKTRPREEQFASTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 13 - protein, 17 aa, Canis lupus
  source 1..17: note = Canis familiaris
FNECRCTDTPPCPVPEP

SEQ ID NO: 14 - protein, 22 aa, Canis lupus
  VARIANT 19: note = X can be either a threonine or an alanine
  source 1..22: note = Canis familiaris
PKRENGRVPRPPDCPKCPXPEM

SEQ ID NO: 15 - protein, 20 aa, Canis lupus
  source 1..20: note = Canis familiaris
AKECECKCNCNNCPCPGCGL

SEQ ID NO: 16 - protein, 17 aa, Canis lupus
  source 1..17: note = Canis familiaris
PKESTCKCISPCPVPES

SEQ ID NO: 17 - protein, 497 aa, Canis lupus
  source 1..497: note = Canis familiaris
VLPAKPENISCIFYYEENFTCTWSPEKEASYTWYKVKRTYSYGYKSDICSTDNSTRGNHASCSFLPPTITNPDNYTIQVEAQNADGIMKSDITYWNLDAIMKIEPPEIFSVKSVLGIKRMLQIKWIRPVLAPHSSTLKYTLRFRTINSAYWMEVNFTKEDIDRDETYNLTELQAFTEYVMTLRCAPAESMFWSGWSQEKVGTTEEEAPYGLDLWRVLKPAMVDGRRPVQLMWKKATGAPVLEKALGYNIWYFPENNTNLTETVNTTNQTHELYLGGKTYWVYVVSYNSLGESPVATLRIPALNEKTFQCIEAMQACLTQDQLVVEWQSSAPEVDTWMVEWFPDVDSEPSSFSWESVSQARNWTIQKDELKPLWCYNISVYPVLRDRVGQPYSTQAYVQEGIPSAGPVTQADSIGVKTVTITWKEIPKSKRNGFIKNYTIFYQAEDGKEFSKTVNSNILQYRLESLTRRTSYSLQVMASTNAGGTNGTKINFKTLSIS

SEQ ID NO: 18 - protein, 317 aa, Canis lupus
  source 1..317: note = Canis familiaris
SMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKET

SEQ ID NO: 19 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 20 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHFHYTQESLSHSPGK

SEQ ID NO: 21 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALLNHYTQESLSHSPGK

SEQ ID NO: 22 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALKNHYTQESLSHSPGK

SEQ ID NO: 23 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHTTQESLSHSPGK

SEQ ID NO: 24 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALLYHYTQESLSHSPGK

SEQ ID NO: 25 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALKYHYTQESLSHSPGK

SEQ ID NO: 26 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALKFHYTQESLSHSPGK

SEQ ID NO: 27 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALLFHYTQESLSHSPGK

SEQ ID NO: 28 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALKYHTTQESLSHSPGK

SEQ ID NO: 29 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALLYHTTQESLSHSPGK

SEQ ID NO: 30 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALLFHTTQESLSHSPGK

SEQ ID NO: 31 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALKFHTTQESLSHSPGK

SEQ ID NO: 32 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPPGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 33 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARDPEVTCVVVDLDPEDPEVQISWFVDGKQMQTAKTQPREEQFNGTYRVVSVLPPGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 34 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARDPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 35 - protein, 573 aa, synthetic construct
  REGION 1..573: note = Modified canine
MGWSCIILFLVATATGVHSSMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHTTQESLSHSPGK

SEQ ID NO: 36 - protein, 478 aa, synthetic construct
  REGION 1..478: note = Modified canine
MGWSCIILFLVATATGVHSEVTLQESGPGLVKPSQTLSLTCSFSGFSLSTFGRGVGWIRQRPGRGLEWMGHIWWDDDKYYNPALKSRLSITKDTAKNQVFLQLSSMTTEDTAVYYCARIAGGLRRAPYAMDSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHFHYTQESLSHSPGK

SEQ ID NO: 37 - protein, 478 aa, synthetic construct
  REGION 1..478: note = Modified canine
MGWSCIILFLVATATGVHSEVTLQESGPGLVKPSQTLSLTCSFSGFSLSTFGRGVGWIRQRPGRGLEWMGHIWWDDDKYYNPALKSRLSITKDTAKNQVFLQLSSMTTEDTAVYYCARIAGGLRRAPYAMDSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 38 - protein, 478 aa, synthetic construct
  REGION 1..478: note = Modified canine
MGWSCIILFLVATATGVHSEVTLQESGPGLVKPSQTLSLTCSFSGFSLSTFGRGVGWIRQRPGRGLEWMGHIWWDDDKYYNPALKSRLSITKDTAKNQVFLQLSSMTTEDTAVYYCARIAGGLRRAPYAMDSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALKFHYTQESLSHSPGK

SEQ ID NO: 39 - protein, 478 aa, synthetic construct
  REGION 1..478: note = Modified canine
MGWSCIILFLVATATGVHSEVTLQESGPGLVKPSQTLSLTCSFSGFSLSTFGRGVGWIRQRPGRGLEWMGHIWWDDDKYYNPALKSRLSITKDTAKNQVFLQLSSMTTEDTAVYYCARIAGGLRRAPYAMDSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALKYHYTQESLSHSPGK

SEQ ID NO: 40 - protein, 478 aa, synthetic construct
  REGION 1..478: note = Modified canine
MGWSCIILFLVATATGVHSEVTLQESGPGLVKPSQTLSLTCSFSGFSLSTFGRGVGWIRQRPGRGLEWMGHIWWDDDKYYNPALKSRLSITKDTAKNQVFLQLSSMTTEDTAVYYCARIAGGLRRAPYAMDSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALLFHYTQESLSHSPGK

SEQ ID NO: 41 - protein, 478 aa, synthetic construct
  REGION 1..478: note = Modified canine
MGWSCIILFLVATATGVHSEVTLQESGPGLVKPSQTLSLTCSFSGFSLSTFGRGVGWIRQRPGRGLEWMGHIWWDDDKYYNPALKSRLSITKDTAKNQVFLQLSSMTTEDTAVYYCARIAGGLRRAPYAMDSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALLYHYTQESLSHSPGK

SEQ ID NO: 42 - protein, 478 aa, synthetic construct
  REGION 1..478: note = Modified canine
MGWSCIILFLVATATGVHSEVTLQESGPGLVKPSQTLSLTCSFSGFSLSTFGRGVGWIRQRPGRGLEWMGHIWWDDDKYYNPALKSRLSITKDTAKNQVFLQLSSMTTEDTAVYYCARIAGGLRRAPYAMDSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLYIARDPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 43 - protein, 478 aa, synthetic construct
  REGION 1..478: note = Modified canine
MGWSCIILFLVATATGVHSEVTLQESGPGLVKPSQTLSLTCSFSGFSLSTFGRGVGWIRQRPGRGLEWMGHIWWDDDKYYNPALKSRLSITKDTAKNQVFLQLSSMTTEDTAVYYCARIAGGLRRAPYAMDSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHHHYTQESLSHSPGK

SEQ ID NO: 44 - protein, 124 aa, synthetic construct
  REGION 1..124: note = Modified canine
EVTLQESGPGLVKPSQTLSLTCSFSGFSLSTFGRGVGWIRQRPGRGLEWMGHIWWDDDKYYNPALKSRLSITKDTAKNQVFLQLSSMTTEDTAVYYCARIAGGLRRAPYAMDSWGQGTLVTVSS

SEQ ID NO: 45 - protein, 217 aa, synthetic construct
  REGION 1..217: note = Modified canine
DIVMTQTPLSLSVSPGETASISCRASENIYSSLAWFRQKPGQSPQLLVYAATNLADGVPDRFSGSGSGTDYTLRISRVEADDTGVYYCQHFRDTPPTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 46 - protein, 5 aa, synthetic construct
  REGION 1..5: note = C-terminal linker
GGGGS

SEQ ID NO: 47 - protein, 19 aa, synthetic construct
  REGION 1..19: note = Signaling peptide
MGWSCIILFLVATATGVHS

SEQ ID NO: 48 - protein, 354 aa, Canis lupus
  source 1..354: note = Canis familiaris
MGVPRPRSWGLGFLLFLLPTLRADSHLSLLYHLTAVSAPPPGTPAFWASGWLGPQQYLSYNNLRAQAEPYGAWVWENQVSWYWEKETTDLRTKEGLFLEALKALGDGGPYTLQGLLGCELGPDNTSVPVAKFALNGEDFMTFDPKLGTWNGDWPETETVSKRWMQQAGAVSKERTFLLYSCPQRLLGHLERGRGNLEWKEPPSMRLKARPGSPGFSVLTCSAFSFYPPELQLRFLRNGLAAGSGEGDFGPNGDGSFHAWSSLTVKSGDEHHYRCLVQHAGLPQPLTVELESPAKSSVPVVGIVIGFLLLTAVAVGGALLWRRMRKGLPAPWMSLRGDDVGALLPTPGVPKDADS

SEQ ID NO: 49 - protein, 125 aa, Canis lupus
  source 1..125: note = Canis familiaris
MAPRPALATAGFLALLLILLAACRLDAVQHPPKIQVYSRHPAENGKPNFLNCYVSGFHPPEIEIDLLKNGKEMKAEQTDLSFSKDWTFYLLVHTEFTPNEQDEFSCRVKHVTLSEPQIVKWDRDN

SEQ ID NO: 50 - protein, 219 aa, Felis catus
PPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 51 - protein, 219 aa, synthetic construct
  REGION 1..219: note = Modified feline
PPEMLGGPSIFIFPPKPKDTLSISRTPEVTCLVVALGPDDSDVQITWFVDNTQVYTAKTSPREEQFASTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 52 - protein, 221 aa, Felis catus
CPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 53 - protein, 221 aa, Felis catus
CPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 54 - protein, 216 aa, Homo sapiens
PELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 216 aa, synthetic construct
  REGION 1..216: note = consensus sequence
  VARIANT 18: note = X can be any naturally occurring amino acid
  VARIANT 106: note = X can be any naturally occurring amino acid
  VARIANT 111: note = X can be any naturally occurring amino acid
  VARIANT 113: note = X can be any naturally occurring amino acid
  VARIANT 128: note = X can be any naturally occurring amino acid
  VARIANT 158: note = X can be any naturally occurring amino acid
LGGPSVFIFPPKPKDTLXISRTPEVTCVVVDLGPEDPEVQISWFVDGKEVHTAKTQPREEQFNSTYRVVSVLPILHQDWLKGKEFKCKVNNKALPSPIERTISKAXGQAHXPXVYVLPPSREELSKNXTVSLTCLIKDFFPPDIDVEWQSNGQPEPEXKYRTTPPQLDEDGSYFLYSKLSVDKSRWQRGDTFTCAVMHEALHNHYTQKSLSHSPGK